(19) United States
(12) Patent Application Publication
(10) Pub. No.: US 2021/0386754 A1
(43) Pub. Date: Dec. 16, 2021

(54) COMPOSITIONS AND METHODS FOR TREATING ATHEROSCLEROTIC VASCULAR DISEASE

(71) Applicant: AUGUSTA UNIVERSITY RESEARCH INSTITUTE, INC., Augusta, GA (US)

(72) Inventor: Gábor CSÁNYI, Martinez, GA (US)

(73) Assignee: AUGUSTA UNIVERSITY RESEARCH INSTITUTE, INC., Augusta, GA (US)

(21) Appl. No.: 17/191,398

(22) Filed: Mar. 3, 2021

Related U.S. Application Data
(60) Provisional application No. 62/984,536, filed on Mar. 3, 2020.

(51) Int. Cl. A61K 31/55; A61K 45/06; A61P 9/10

(52) U.S. Cl. CPC A61K 31/55; A61P 9/10; A61K 45/06

(57) ABSTRACT

One embodiment provides a method for treating atherosclerotic vascular disease in a subject in need thereof by administering to the subject an effective amount of a pharmaceutical composition comprising one or more inhibitors of macropinocytosis to inhibit or reduce receptor-independent LDL macropinocytosis in the subject. In one embodiment, the one or more inhibitors of macrophage macropinocytosis is imipramine or a derivative thereof or any additional macropinocytosis inhibitors independent of their effects on NHE1 activity. In some embodiments the macropinocytosis is macrophage macropinocytosis. A representative atherosclerotic vascular disease is atherosclerosis.

### CROSS-REFERENCE TO RELATED APPLICATIONS

This application claims priority to and benefit of U.S. Provisional Patent Application No. 62/984,536 filed on Mar. 3, 2020, and which is incorporated by reference in its entirety.

### STATEMENT REGARDING FEDERALLY SPONSORED RESEARCH

This invention was made with government support under R00HL114648 awarded by the National Institutes of Health. The government has certain rights in the invention.

### FIELD OF THE INVENTION

This invention is generally related to compositions and methods of treating atherosclerotic vascular diseases.

### BACKGROUND OF THE INVENTION

Atherosclerotic vascular disease is the underlying cause of myocardial infarction, ischemic stroke, sudden cardiac death, stable and unstable angina, and peripheral artery disease. The development of atherosclerosis is initiated by subendothelial retention of plasma-derived apolipoprotein B (apoB)-containing lipoproteins (e.g., LDL) in focal areas of arteries, particularly regions in which laminar flow is disturbed by bends and at bifurcations. Subendothelial macrophages internalize LDL lipoproteins in the arterial wall, become lipid-laden foam cells that may undergo apoptosis and, if not coupled with efferocytic clearance, contribute to the formation and expansion of atherosclerotic lesions. Advanced atherosclerotic lesions may undergo destabilization, leading to rupture, thrombosis and compromised oxygen supply of affected organs.

Macrophage uptake of modified LDL [e.g., acetylated (ac-) and oxidized (ox-) LDL] by scavenger receptors (SR) has become the most widely accepted mechanism for macrophage foam cell formation during the last three decades. Molecular cloning identified SR family members CD36 and SR-A as high-affinity receptors for ac-LDL and ox-LDL and subsequent binding studies confirmed that deletion of CD36 and SR-A in macrophages (CD36^{-/-}/SR-A^{-/-}) inhibits 80% and 90% uptake of ac-LDL and ox-LDL, respectively. Despite the dominance of this paradigm, several studies have demonstrated a partial or lack of inhibition of the lesion area in hypercholesterolemic CD36^{-/-}, SR-A^{-/-} and CD36^{-/-}/SR⁻A^{-/-} mice. Unexpectedly, clinical studies reported an increased mortality rate from atherosclerotic coronary artery disease (CAD) in CD36-deficient patients and most clinical trials failed to support benefits of antioxidant therapies in patients with atherosclerotic vascular disease. Although there are many potential and speculative explanations why antioxidants failed to improve clinical outcomes in patients with cardiovascular disorders, they also support the existence of a yet uncharacterized, SR-independent cellular mechanisms of lipid uptake in atherosclerotic vessels. Supporting this statement, in vitro studies by Kruth et al. have offered alternative, LDL receptor (LDLR)- and SR-independent mechanisms of macrophage lipoprotein uptake via fluid-phase macropinocytosis. These studies demonstrated that stimulation of macrophage macropinocytosis in the presence of exogenous unmodified, native LDL (nLDL) at concentrations considered within the normal range (<100 mg/dl) promotes foam cell formation in vitro. Growth factors (PDGF, EGF, M-CSF) and inflammatory cytokines (IFN-γ and TNF-α) have been reported to stimulate macrophage macropinocytosis in vitro. Mechanistically, it was found that growth factors and inflammatory cytokines promote activation of their downstream effectors, Ras and PI3 kinase (PI3K), leading to dynamic phosphorylation of phosphatidylinositols, submembranous actin polymerization, membrane ruffling, macropinosome formation and macropinocytosis. Importantly, the levels of growth factors and inflammatory cytokines found to stimulate macrophage macropinocytosis in vitro are elevated in atherosclerotic arteries in vivo. In addition, cholesterol levels found in human arteries saturate SR-mediated macrophage LDL uptake, thus further supporting the role of receptor-independent uptake mechanisms of lipids in atherosclerotic vessels. Despite this information, no previous studies have inhibited macropinocytosis selectively in myeloid cells in animal models of atherosclerosis, investigated the contribution of SR-vs. macropinocytosis-mediated LDL uptake to atherosclerosis development, quantified macrophage lipid macropinocytosis in atherosclerotic arteries, or analyzed human and murine atherosclerotic vessels for evidence of macropinocytosis-associated plasma membrane activities.

The most effective therapy to date that reduces atherosclerotic cardiovascular disease works by decreasing plasma LDL levels, thereby reducing the likelihood that these cholesterol rich particles will enter the arterial wall and become internalized by subendothelial macrophages. Despite the currently available drug-based therapies and advanced surgical interventions, atherosclerotic cardiovascular disease accounts for majority of deaths in the Western world and its therapeutic management remains one of the most serious challenges in cardiovascular medicine.

Therefore, it is an object of the invention to provide compositions and methods for the treatment or prevention of atherosclerotic vascular disease.

It is an object of the invention to provide compositions and methods for the treatment of atherosclerotic vascular disease and other diseases involving macrophage macropinocytosis.

### SUMMARY OF THE INVENTION

Disclosed herein are compositions and methods useful for the treatment or prevention of atherosclerotic vascular disease and other diseases involving macrophage macropinocytosis in a subject in need thereof. Some embodiments provide a clinically relevant inhibitor of macrophage macropinocytosis that lacks regulatory effects on NHE1 and tests its ability to inhibit atherosclerosis development.

One embodiment provides a method for treating atherosclerotic vascular disease in a subject in need thereof by administering to the subject an effective amount of a pharmaceutical composition comprising one or more inhibitors of macropinocytosis to inhibit or reduce receptor-independent LDL macropinocytosis in the subject. In one embodiment, the one or more inhibitors of macrophage macropinocytosis is 5-(N-ethyl-N-isopropyl)amiloride, imipramine or derivatives thereof or any additional macropinocytosis inhibitors independent of their effects on NHE1 activity. In some embodiments the macropinocytosis is macrophage macropinocytosis. A representative atherosclerotic vascular disease is atherosclerosis.

Another embodiment provides a method for reducing plaque formation in a subject's circulatory system in need thereof by administering to the subject an effective amount of a pharmaceutical composition comprising one or more inhibitors of macropinocytosis to inhibit or reduce receptor-independent LDL macropinocytosis, reduce plaque formation or induce atherosclerosis regression in the subject's circulatory system. In some embodiments the one or more inhibitors of macrophage macropinocytosis is imipramine or a derivative thereof or any additional macropinocytosis inhibitors independent of their effects on NHE1 activity. In some embodiments the macropinocytosis is macrophage macropinocytosis. In some embodiments, the subject has atherosclerosis.

Still another embodiment provides a method for reducing or inhibiting foam cell formation in a subject in need thereof by administering to the subject an effective amount of a pharmaceutical composition comprising one or more inhibitors of macropinocytosis to inhibit or reduce receptor-independent LDL macropinocytosis to inhibit or reduce foam cell formation in the subject. In some embodiments, the one or more inhibitors of macrophage macropinocytosis comprises imipramine or a derivative thereof or any additional macropinocytosis inhibitors independent of their effects on NHE1 activity. In some embodiments the macropinocytosis is macrophage macropinocytosis. In some embodiments, the subject has atherosclerosis.

Yet another embodiment provides a method for treating atherosclerotic vascular disease in a subject in need thereof by administering to the subject an effective amount of a pharmaceutical composition comprising one or more inhibitors of macropinocytosis to inhibit or reduce receptor-independent LDL macropinocytosis in the subject in combination or alternation with one or more statins, other lipid lowering agents or cardiovascular therapeutics. In one embodiment the one or more inhibitors of macrophage macropinocytosis comprises imipramine or a derivative thereof or any additional macropinocytosis inhibitors independent of their effects on NHE1 activity and the one or more statins are selected from the group consisting of atorvastatin, fluvastatin, lovastatin, pravastatin, rosuvastatin, simvastatin, pitavastatin, and combinations thereof.

Still another embodiment provides a pharmaceutical composition including an effective amount of one or more inhibitors of macropinocytosis and an effective amount of one or more statins, other lipid lowering agents or cardiovascular therapeutics. In one embodiment, the one or more inhibitors of macrophage macropinocytosis comprises imipramine or a derivative thereof or any additional macropinocytosis inhibitors independent of their effects on NHE1 activity and the one or more statins are selected from the group consisting of atorvastatin, fluvastatin, lovastatin, pravastatin, rosuvastatin, simvastatin, pitavastatin, and combinations thereof. In some embodiments the pharmaceutical composition is formulated for oral administration.

One embodiment provides compositions and methods that inhibit of macrophage macropinocytosis of LDL to promote a decrease of atherosclerotic lesion development. In another embodiment, the disclosed compositions are pharmaceutical composition including inhibitory agents that inhibit intracellular LDL accumulation in macrophages of subjects with atherosclerotic vascular disease. In one embodiment the inhibitory agent inhibits LDL macropinocytosis in macrophages. In a preferred embodiment the inhibitory agent is imipramine also known as Tofranil™ or any additional macropinocytosis inhibitors independent of their effects on NHE1 activity.

In one embodiment the inhibitory agent inhibits LDL macropinocytosis in macrophages to reduce the generation of macrophage foam cells. Another embodiment provides inhibitory agents that reduce the generation of macrophage foam cells. In an additional embodiment the disclosed inhibitory agent induces, promotes, or enhances the reduction of atherosclerotic plaques in a subject with atherosclerotic vascular disease. In another embodiment the disclosed inhibitory agent reduces or prevents the formation of atherosclerotic plaques in a subject in need thereof.

In some embodiments, atherosclerotic vascular disease is characterized by high levels of circulating LDL relative to subjects without the disease or condition. In some embodiments, atherosclerotic vascular disease is characterized by increased macrophage macropinocytosis of LDL. In additional embodiments, atherosclerotic vascular disease is characterized by increased formation of macrophage foam cells. In another embodiment, atherosclerotic vascular disease is characterized by the formation of atherosclerotic plaques in the arterial wall. One embodiment provides a method for treating atherosclerotic vascular disease in a subject in need thereof by administering to the subject an effective amount of a pharmaceutical composition including inhibitors of macrophage macropinocytosis.

Another embodiment provides a method for treating atherosclerotic vascular disease in a subject in need thereof by administering to the subject the combination of effective amount of a pharmaceutical composition including LDL lowering therapies and inhibitors of macrophage macropinocytosis to lower circulating LDL levels, improve the barrier function of endothelial cells to attenuate LDL uptake into the arterial wall and inhibit macrophage internalization of LDL for the treatment atherosclerotic vascular disease.

### BRIEF DESCRIPTION OF THE DRAWINGS

FIGS. 1A-1J show the macropinocytosis inhibitor EIPA reduces atherosclerosis development in wild-type and SR knockout mice. FIGS. 1A to 1E show male WT and CD36^{-/-}SR-A^{-/-} mice treated±EIPA, PCSK9-AAV+partial LCA ligation and fed a Western diet for 4 weeks. FIG. 1A provides representative images of isolated LCA (arrows), scale bar: 1 mm. FIG. 1B shows Oil Red O (ORO) staining for proximal, middle and distal LCA segments, scale bar: 100 μm. FIG. 1C shows the quantification of ORO positive area (n=11, 13, 8 and 8 for WT±EIPA, CD36^{-/-}SR-A^{-/-}±EIPA, respectively). FIG. 1D provides representative images of H&E, Masson's trichrome and CD68 staining of LCA. FIGS. 1E to 1G show quantification of collagen deposition (1E), lesion area (1F) and CD68⁺ macrophages (1G) in the LCA (n=5). FIG. 1H shows total plasma cholesterol levels (n=11, 13, 9 and 9 for WT+vehicle, WT+EIPA, CD36^{-/-} SR-A^{-/-}+vehicle and CD36^{-/-}SR-A^{-/-}+EIPA group, respectively). FIG. 1I shows body weight prior to sacrifice (n=9). FIG. 1J shows systolic blood pressure (n=5). Data are mean±SEM. *p<0.05 vs. vehicle.

FIGS. 2A to 2M show the characterization of lipid macropinocytosis by WT and CD36^{-/-}SR-A^{-/-} macrophages in vitro. FIG. 2A is schematic diagram illustrating morphological plasma membrane changes during macropinocytosis. FIG. 2B to 2E show wild-type and CD36^{-/-}SR-A^{-/-} BMDM overexpressing Hras^{G12V} or GFP control, ±EIPA (25 μM, 1 hr) treatment. FIG. 2B provides representative SEM images. Arrows point to—linear ruffles; curved ruffles or macropinocytic cups, scale bar 1 μm. FIG. 2C shows the quantification of membrane ruffles (n=3). FIG. 2D shows Hras^{G12V} overexpressing and GFP control macrophages incubated with 50 μg/mL nLDL for 24 hr. Representative images (n=3) of ORO staining, scale bar 50 μm. FIG. 2E shows cells were treated as described in (2D) and Nile Red fluorescence was quantified by FACS (n=4). FIG. 2F shows THP-1 macrophages were treated with PMA (1 μM) to stimulate macropinocytosis and incubated with Dil-nLDL (0, 50, 100 and 150 μg/mL) for 24 hr. DiI fluorescence analyzed by FACS (n=3). FIG. 2G shows macropinocytosis of extracellular solutes (arrows Alexa Fluor 488-dextran), macropinosome formation (FM 4-64 [plasma membrane marker], bottom arrows) and membrane ruffling—top arrows. Scale bar: 5 m. FIGS. 2H and 21 show wild-type BMDM treated with 100 ng/mL M-CSF for 24 hours. Plasma membrane SR-A (2H) and CD36 (2I) expression analyzed by FACS (n=4). FIGS. 2J and 2K are schematic diagrams illustrating macropinosome-mediated SR internalization. FIGS. 2L and 2M show the mathematical description of LDL macropinocytosis (2L) and scavenger receptor-mediated lipid endocytosis (2M) in atherosclerotic arteries. Data are mean±SEM. *p<0.05 vs. vehicle, ^{#}p<0.05 vs. MCSF or PDGF.

FIGS. 3A to 3I show that physiologically relevant stimulation of macropinocytosis promotes lipid accumulation in wild-type and SR knockout macrophages. FIGS. 3A and 3B show the analysis of publically available database demonstrating increased expression of M-CSF (3A) and PDGF (3B) in human atherosclerotic arteries compared with control tissue (n=32). FIG. 3C shows M-CSF (100 ng/mL) and PDGF (200 ng/mL) stimulated nLDL internalization in THP-1 macrophages via macropinocytosis (n=4). FIG. 3D shows M-CSF- and PDGF-induced nLDL (50 μg/mL) internalization is impaired in NHE1-deficient BMDM (n=4). FIG. 3E shows M-CSF and PDGF stimulate cholesterol accumulation in WT and CD36^{-/-}SR-A^{-/-} BMDM incubated with nLDL (50 μg/mL) (n=5). FIG. 3F shows THP-1 macrophages treated with vehicle, 100 ng/mL M-CSF or 200 ng/mL PDGF in the presence of 50 μg/mL nLDL for 24 hours. LDL oxidation was determined by agarose gel electrophoresis of collected conditioned media. Positive controls: nLDL+CuSO₄ (50 μM, 72 hr) and ox-LDL. Negative control: nLDL. FIG. 3G shows the quantification of electrophoretic mobility (n=3). FIGS. 3H and 3I show THP-1 macrophages that were pretreated with ±SOD (100 U/mL) and/or catalase (250 U/mL) for 1 hr, incubated with 50 μg/mL nLDL for 24 hours in the presence or absence of M-CSF (3H) or PDGF (3I). Cells were incubated with 50 ng/mL Nile Red for 7 min and FACS quantification for Nile Red fluorescence was performed. Data are mean±SEM. *p<0.05 vs. vehicle, ^{#}p<0.05 vs. M-CSF or PDGF.

FIGS. 4A to 4K show the visualization of macrophage macropinocytosis in human and murine atherosclerotic arteries. FIG. 4A shows the serial section TEM imaging demonstrating the presence of plasma membrane protrusions on the surface of lipid-laden macrophages in atherosclerotic ApoE^{-/-} aorta. LD: lipid droplets, N: nucleus. Arrows—membrane protrusion, asterisk—curving ruffle, arrows—parallel side of identified ruffle. Scale bar, 1 μm. FIG. 4B is a 3D reconstruction of a foamy macrophage in ApoE^{-/-} aorta. Scale cube, 1 μm³. FIG. 4C shows the calculated total surface area, ruffle volume and tip-base distance of identified membrane ruffles. FIG. 4D is a 3D reconstruction of curved ruffle. Inset: TEM image of curved ruffle in 2D. H=height, W=width and D=depth. FIG. 4E shows human heart and aortic tissue isolated from a cadaveric donor with a history of cardiovascular disease (patient #1). IC=atheroprone Inner Curvature. FIGS. 4F and 4G show TEM imaging demonstrating formation of single membrane protrusions (arrows), parallel ruffles (asterisk) and membrane-derived vesicles (arrows) in IC segments of human atherosclerotic aorta. FIG. 4H left panel shows the cross section of human atherosclerotic LAD (patient #2). FIG. 4H right panel shows immunogold-labeling of LDL (arrows). Open cups—arrows, macropinosomes: arrows. FIG. 4I is a schematic diagram illustrating the design of in vivo LDL tracking experiments. FIG. 4J shows a representative gating strategy identifying isolated F4/80⁺ macrophages from atherosclerotic ApoE^{-/-} aorta. FACS analysis of DiI fluorescence in isolated F4/80⁺ cells. FIG. 4K shows the quantification of mean fluorescence intensity, n=3, *p<0.05. Data are mean±SEM.

FIGS. 5A to 5L show that genetic deletion of NHE1 in myeloid cells reduces atherosclerosis development in hypercholesterolemic mice. In FIGS. 5A to 5J, NHE1^{f/f} and NHE1^{ΔM} mice were injected with PCSK9-AAV, subjected to partial LCA ligation and fed a Western diet for 3 weeks to induce atherosclerosis. FIG. 5A shows representative images of isolated LCA (arrows), scale bar: 1 mm. FIG. 5B shows Oil Red O staining for proximal, middle and distal LCA segments, scale bar: 100 μm. FIG. 5C shows the quantification of ORO positive area (n=15 and 13 for NHE1^{f/f} and NHE1^{ΔM}, respectively). FIG. 5D shows total cholesterol levels. FIG. 5E shows lipid profile of pooled serum (n=5). FIG. 5F shows blood glucose levels (n=9 and 7 for NHE1^{f/f} and NHE1^{ΔM} mice, respectively). FIG. 5G shows the systolic blood pressure (n=5 and 4 for NHE1^{f/f} and NHE1^{ΔM} mice, respectively). FIG. 5H shows the body weight measured prior to sacrifice (n=9 and 7 for NHE1^{f/f} and NHE1^{ΔM} mice, respectively). FIG. 5I shows the fat mass and FIG. 5J shows the fluid content measured using NMR (n=9 and 7 for NHE1^{f/f} and NHE1^{ΔM} mice, respectively). FIGS. 5K and 5L show the internalization of Dil-nLDL in NHE1^{f/f} and NHE1^{ΔM} peritoneal macrophages. Confocal images of isolated macrophages (5K), FACS analysis of Dil fluorescence (5L) (n=5). Data are mean±SEM. *p<0.05.

FIGS. 6A to 6L show that the genetic deletion of NHE1 in myeloid cells inhibits atherosclerosis development in the aortic root and thoracic aorta of LDLR-deficient mice fed a Western diet for 16 weeks. FIG. 6A shows enface staining with Oil Red O in the thoracic aorta. Scale bar: 2 mm. FIG. 6B provides representative images of Oil Red O, H&E and Masson's trichrome staining of the aortic sinus. Scale bar 0.2 mm. FIG. 6C shows the quantification of aortic Oil Red O positive area in (6A). FIGS. 6D to 6F show the quantification of Oil Red O positive area (6D), lesion area (6E) and collagen deposition (6F) in the aortic sinus (n=9 and 10 for NHE1^{f/f} and NHE1^{ΔM}, respectively). FIG. 6G shows the total cholesterol levels. FIG. 6H shows the systolic blood pressure. FIG. 6I shows the body weight (n=9 and 10 for NHE1^{f/f} and NHE1^{ΔM} respectively). FIG. 6J shows the fat mass (n=9 and 10 for NHE1^{f/f} and NHE1^{ΔM}, respectively. FIG. 6K shows the fluid content (n=9 and 10 for NHE1^{f/f} and NHE1^{ΔM}, respectively). FIG. 6L shows blood glucose levels. mean±SEM. *p<0.05.

FIGS. 7A to 7O show that treatment with a “repurposed” FDA-approved drug that inhibits macropinocytosis attenuates atherosclerosis development in hypercholesterolemic mice. FIG. 7A shows THP-1 macrophages were pretreated with 5 μM imipramine for 1 hour, then treated with vehicle, 100 ng/mL M-CSF or 200 ng/mL PDGF in the presence of 50 μg/mL nLDL for 24 hours. Cells were incubated with 50 ng/mL Nile Red for 7 min and FACS quantification for Nile Red fluorescence was performed (n=4). In FIGS. 7B to 7O Wild-type mice were injected with PCSK9-AAV, underwent partial LCA ligation and fed a Western diet for 4 weeks to induce atherosclerosis. Mice were treated with vehicle or imipramine (sc). FIG. 7B shows representative images of LCA (arrows), scale bar: 1 mm. FIG. 7C shows representative Orcein and Martius Scarlet Blue (OMSB) staining for proximal, middle and distal segments and Segmentation algorithm of LCA. L: lumen, P: plaque area, VWA: Vessel wall area. Scale bar: 100 μm (Red). The inset shows zoomed-in images for distal segments of LCA, scale bar: 50 μm (Black). FIG. 7D shows the quantification of plaque area (n=5). FIG. 7E area under the curve (AUC). FIGS. 7F to 7I show the quantification of plaque area (7F), vessel wall area (7G), relative internal vessel area (IVA %) (7H) and relative collagen area (Collagen %) (7I) (n=5). FIG. 7J shows the blood glucose (n=9). FIG. 7K shows the systolic blood pressure (n=5). FIG. 7L shows the body weight (n=9). FIG. 7M shows the fat mass (n=9) and FIG. 7N shows the fluid content (n=9). FIG. 7O shows the total cholesterol levels (n=9). Data are mean±SEM. *p<0.05 vs. vehicle, ^{#}p<0.05 vs. M-CSF or PDGF.

FIGS. 8A to 81 show that EIPA treatment inhibits macrophage macropinocytosis and generation of CD36^{-/-}SR-A^{-/-} mice. FIG. 8A shows pre-incubation of THP-1 macrophages with EIPA (25 μM, 1 hr) inhibits PMA-induced nLDL (50 μg/ml) macropinocytosis (n=5). FIGS. 8B, 8C, and 8D show EIPA (25 μM, 1 hr) does not inhibit SR-mediated ox-LDL (50 μg/ml) uptake, clathrin-[FITC-transferrin (1 μg/ml)], and caveolin-mediated [AF488-albumin (1 μg/ml)] endocytosis (n=3). In FIG. 8E macrophages were preincubated with EIPA (25 μM, 1 hr), then treated with PMA for 4 hours. Macrophages incubated with 70% ethanol for 30 seconds were used as positive controls. Cells were stained with the LIVE/DEAD™ Fixable Far Red dye to identify live cells (n=3). FIG. 8F is an agarose gel showing representative genotyping experiments. FIGS. 8G and 8H show CD36 (n=6) and SR-A (n=5) mRNA expression in WT and CD36^{-/-}SR-A^{-/-} BMDM. FIG. 8I shows WT and CD36^{-/-}SR-A^{-/-} BMDM were treated with 50 μg/mL nLDL or ox-LDL for 24 hr. Nile Red fluorescence was quantified using flow cytometry (n=6). Data are mean±SEM. *p<0.05 vs. vehicle or WT, ^{#}p<0.05 vs. PMA.

FIGS. 9A to 9C show that EIPA does not inhibit monocyte adhesion to human aortic endothelial cells in vitro. CFDA-labeled THP-1 monocytes were treated±EIPA (25 μM, 1 hr) and seeded on the surface of confluent, deep red cell tracker-labeled HAEC. HAEC were pretreated with vehicle or TNFα (10 ng/ml, 4 hr) to promote monocyte adhesion. After extensive washing, cells were imaged using a Zeiss 780 inverted confocal microscope. FIG. 9A provides representative images of monocyte adhesion. FIG. 9B shows the quantitative data showing number of monocytes adhered to the surface of endothelial cells. Scale bar=50 μm. (n=3). FIG. 9C shows qRT-PCR analysis of SR-B1 mRNA expression in HAEC treated with ±EIPA (25 μM, 24 hr) (n=4). Data are mean±SEM.

FIGS. 10A to 10G show that EIPA treatment attenuates atherosclerosis in male ApoE^{-/-} mice. Male ApoE^{-/-} mice underwent partial LCA ligation, fed a Western diet for 4 weeks and received vehicle or EIPA via a subcutaneously implanted minipump. FIG. 10A provides representative images of isolated LCA (arrows), scale bar: 1 mm. FIG. 10B shows Oil Red O (ORO) staining for proximal, middle and distal LCA segments, scale bar: 100 μm. FIG. 10C shows the quantification of ORO positive area (n=5 and 6 for vehicle and EIPA treatment, respectively). FIG. 10D shows the total plasma cholesterol levels (n=5 and 6 for vehicle and EIPA treatment, respectively). FIG. 10E shows the body weight prior to sacrifice (n=5 and 6 for vehicle and EIPA treatment, respectively). FIG. 10F shows the fat mass measured using NMR (n=5 and 6 for vehicle and EIPA treatment representative). FIG. 10G shows the systolic blood pressure (n=5 and 4 for vehicle and EIPA treatment, respectively). Data are mean±SEM. *p<0.05.

FIGS. 11A to 11G show that EIPA treatment attenuates atherosclerosis in female WT mice. Female WT mice were injected with PCSK9-AAV, underwent partial LCA ligation, and fed a Western diet for 4 weeks, mice were received vehicle or EIPA via a subcutaneously implanted minipump FIG. 11A provides representative images of isolated LCA (arrows), scale bar: 1 mm. FIG. 11B provides representative Orcein and Martius Scarlet Blue (OMSB) staining for proximal, middle and distal segments of LCA. Scale bar: 100 μm. FIG. 11C shows the quantification of plaque area (n=5). FIG. 11D shows the total plasma cholesterol levels (n=9-10). FIG. 11E shows the body weight prior to sacrifice (n=10). FIG. 11F shows the fat mass measured using NMR (n=11). FIG. 11G shows the systolic blood pressure (n=5). Data are mean±SEM. *p<0.05.

FIGS. 12A to 12C show that EIPA inhibits Ras- (HRAS^{G12V}) induced LDL internalization in CD36^{-/-}/SR-A^{-/-} macrophages. FIG. 12A shows CD36^{-/-}/SR-A^{-/-} BMDM overexpressing HRas^{G12V} were pretreated with ±EIPA (25 μM, 1 hr), incubated with 50 μg/mL ox-LDL or ac-LDL for 24 hr. FACS analysis performed to measure Nile red fluorescence. (n=3). FIG. 12B shows Dil-labeled cholesterol efflux from RAW 264.7 macrophages (n=5). FIG. 12C shows RAW 264.7 macrophages were pretreated with ±EIPA (25 μM, 1 hr), incubated with 50 μg/mL Dil-LDL for 24 hr. FACS analysis performed to measure Nile red fluorescence (n=4). Data are mean±SEM. *p<0.05 vs. vehicle, ^{#}p<0.05 vs. PMA or AD Hras^{G12V}.

FIGS. 13A and 13B show confocal images for vehicle- and PMA-treated cells. Quantification of macrophage membrane ruffling, cup formation and closed cup in ApoE^{-/-} atherosclerotic arteries. FIG. 13A shows macropinocytosis of extracellular solutes (Alexa Fluor 488-dextran, arrows), macropinosome formation (−FM 4-64 [plasma membrane marker], arrows) and membrane ruffling—arrows. Scale bar: 5 m. FIG. 13B shows the quantification of macropinocytosis in atherosclerotic arteries (n=2).

FIGS. 14A to 14D show data produced using NHE1 myeloid-cell specific knockout mice. FIG. 14A is an agarose gel showing representative genotyping experiments. FIG. 14B shows qRT-PCR analysis of NHE1 expression in BMDM (n=3). FIG. 14C shows qRT-PCR analysis showing expression of various NHE isoforms in WT BMDM (n=3). Data were normalized to NHE8. FIG. 14D shows qRT-PCR analysis showing expression of various NHE isoforms in NHE1^{f/f} and NHE1^{ΔM} BMDM (n=4). Data were normalized to NHE8. Data are mean±SEM. *p<0.05.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form.

The term “atherosclerotic vascular disease (also referred to as atherosclerosis, arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Inflammation occurs in combination with lipid accumulation in the vessel wall, and vascular inflammation is with the hallmark of atherosclerosis disease process.

The term “combination therapy”, as used herein, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The terms “individual”, “host”, “subject”, and “patient” are used interchangeably herein, and refer to a mammal, including, but not limited to, humans, rodents, such as mice and rats, and other laboratory animals.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%. The terms “further inhibiting”, “further inhibit” or “further inhibition are used herein to refer to reducing the amount or rate of a second process, to stopping the second process entirely, or to decreasing, limiting, or blocking the action or function thereof in addition to reducing the amount or rate of a first process, to stopping the first process entirely, or to decreasing, limiting, or blocking the action or function thereof. The term “inhibitory profile” as used herein refers to the characteristic pattern of reduction of the amount or rate or decrease, blocking or limiting of the action of more than one protein or enzyme. The terms “substantially inhibiting”, “substantially inhibit”, “substantially inhibited”, or “substantially inhibition” are used here to refer to inhibition of kinase activity by at least 65%.

The term “macropinocytosis” refers to a form of endocytosis that allows for the regulated internalization of extracellular solute molecules.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “reduced” or “to reduce” or “reducing” as used herein refer to a diminution, a decrease, an attenuation, limitation or abatement of the degree, intensity, extent, size, amount, density, number or occurrence of disorder in individuals at risk of developing the disorder.

The phrase “subject in need of such treatment” is used to refer to a patient who has or will suffer from or is at risk of developing the disorder.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of retinopathy. A therapeutically effective amount may also refer to the amount of the therapeutic agent that provides a therapeutic benefit in the treatment or management of a disease.

### II. Compositions and Methods for Treating Atherosclerotic Vascular Disease

The decades-old dogma in cardiovascular medicine is that scavenger receptor (SR)-mediated uptake of modified lipoproteins by subendothelial macrophages is the only—or primary—mechanism responsible for deposition of arterial lipids and the development of atherosclerosis. In some embodiments the role of receptor-independent LDL macropinocytosis in arterial lipid accumulation and pathogenesis of atherosclerosis.

Macropinocytosis (a.k.a. fluid-phase endocytosis) is an endocytic mechanism mediating internalization of extracellular fluid and nonspecific bulk uptake of pericellular solutes. Pharmacological blockade of Na⁺—H⁺ exchanger 1 (NHE1) using 5-(N-ethyl-N-isopropyl)amiloride (EIPA) and its analogs is currently regarded as the most effective and selective approach to inhibit macropinocytosis both in vitro and in vivo. However, possible effects of these drugs on transmembrane Na⁺ transport, pH regulation and water homeostasis, in addition to their capacity to block macropinocytosis, limit their use as therapeutic agents in cardiovascular medicine.

The role of SR-mediated modified lipid uptake in macrophage foam cell formation was first described four decades ago (Goldstein et al., Proc Natl Acad Sci USA 76, 333-337 (1979)). These observations have been confirmed by numerous studies and led to the classical dogma that subendothelial modification of LDL plays a critical role in the pathogenesis of atherosclerosis and unmodified lipids are not atherogenic. The present invention presents the role of SR-independent macrophage LDL macropinocytosis in the pathogenesis of atherosclerosis. The rationale for investigating SR- and modification (i.e. oxidation)-independent pathways of LDL uptake is predicated on the observations that i) SR-knockout mice are not or only partially protected from atherosclerosis development, ii) mortality rate from atherosclerotic coronary artery disease is higher in CD36-deficient patients compared to the general population, iii) oxidized LDL is detected only in later stages of atherosclerosis development in human vessels and iv) antioxidant therapies have failed in clinical trial. Although, these results imply the existence of alternative, SR-independent pathway of macrophage lipid internalization in the pathogenesis of atherosclerosis, the precise mechanisms remain poorly understood.

Two studies have investigated macrophage macropinocytosis in atherosclerotic vessels (Buono et al., J Clin Invest 119, 1373-1381 (2009) and Anzinger et al., J Vasc Res 54, 195-199 (2017)). These studies demonstrated that macrophages internalize Angiospark fluorescent nanoparticles in the arterial wall via macropinocytosis. Despite these earlier observations, no previous studies have inhibited macropinocytosis selectively in myeloid cells in animal models of atherosclerosis, investigated macrophage lipid macropinocytosis in atherosclerotic arteries, or analyzed atherosclerotic vessels (human or murine) for evidence of macropinocytosis-associated plasma membrane activities.

While progress has been made in terms of diagnosis, prevention and treatment, atherosclerosis and its cardiovascular consequences continue to cause high rates of morbidity and mortality worldwide. Therefore, there is a need to provide better therapies for inhibiting macrophage lipid macropinocytosis in atherosclerotic vascular disease.

One embodiment provides a method of treating or preventing atherosclerotic vascular disease in a subject in need thereof by inhibiting macrophage macropinocytosis of LDL to decrease atherosclerotic lesion development.

The role of macrophage LDL macropinocytosis in atherosclerosis development is supported by pharmacological studies presented herein in male and female mice, LDLR-knockdown and ApoE^{-/-} mice as well as genetic inhibition of macropinocytosis selectively in myeloid cells. To date, no signaling molecules that selectively or exclusively mediate macropinocytosis have been identified. Inhibition of NHE1 most likely affects pathways other than macropinocytosis. Importantly, the most specific inhibitor currently available was used and combined this with the first genetic mouse model to inhibit macropinocytosis selectively in myeloid (or any other cell types) in vivo. To directly address potential limitations of targeting NHE1, a structurally and mechanistically distinct small molecule compound [FDA-approved drug in a screen of >640 drugs;] that inhibits macropinocytosis (and not other endocytic mechanisms) in macrophages in an NHE1 independent manner was identified. Treatment of mice with imipramine inhibited macrophage macropinocytosis in vitro and attenuated development of atherosclerosis. In one embodiment, macrophage macropinocytosis of LDL is inhibited by administering inhibitory agent that inhibits LDL macropinocytosis in macrophages. In one embodiment the inhibitory agent is imipramine.

The results disclosed herein are consistent with a previous report demonstrating that amiloride reduces lesion development in lipopolysaccharide (LPS)-treated ApoE^{-/-} mice (Y. Zhao et al., Biochem Biophys Res Commun 427, 125-132 (2012)). It is, however, important to add that amiloride inhibits T-type calcium channels, epithelial sodium channels and NHE in multiple cell types and blocks urokinase plasminogen activator activity. As a consequence, amiloride may induce changes in intracellular Ca²⁺ levels, K⁺ concentration, intracellular pH, kidney function, salt-water homeostasis, blood pressure, platelet function and hemostasis in addition to blocking macropinocytosis. The present study targeted macropinocytosis in wild type and SR knockout mice using EIPA and presents an important role for macropinocytosis in macrophage uptake of LDL in vivo. Although CD36 and SR-A mediate 80-90% of modified LDL uptake by macrophages compensatory upregulation of alternative SR-mediated pathways in the model presented herein is possible.

A. Methods for Inhibiting Macrophage Macropinocytosis of LDL in Atherosclerotic Vascular Disease

Methods for inhibiting macrophage macropinocytosis of LDL in atherosclerosis are provided herein. An exemplary method of inhibiting macrophage macropinocytosis of LDL in atherosclerosis includes administering a micropinocytosis inhibitor to a subject in need thereof. In one embodiment the inhibitory agent is imipramine or any additional macropinocytosis inhibitors independent of their effects on NHE1 activity.

The importance of lipid macropinocytosis in atherosclerosis is supported by the results presented herein and the literature demonstrating that a) macropinocytosis of nLDL is linear to extracellular lipid concentration, b) cholesterol levels in the arterial wall is 25-40 folds higher than saturating concentrations of SR-mediated modified lipid uptake, c) macrophages internalize both nLDL and modified LDL via macropinocytosis, d) physiological stimulators of macrophage macropinocytosis (e.g. growth factors, cytokines and matrix proteins) are upregulated in human atherosclerotic arteries and e) stimulation of macropinocytosis decreases plasma membrane expression of SR via their macropinosome-mediated cytosolic internalization. These results indicate that macrophages may play an important role in atherosclerosis by contributing to the initiation of early atherogenesis (nLDL is present) and plaque progression (both nLDL and modified LDL are present). It is important to note that SR-mediated LDL uptake and lipid macropinocytosis are not mutually exclusive, they could contribute to atherosclerosis independently or even regulate each other. Indeed, the examples presented herein suggest that stimulation of macropinocytosis inhibits cell surface expression of SR in macrophages. Pro-inflammatory (M1) and anti-inflammatory (M2) macrophage polarization plays an important role in the pathogenesis of atherosclerosis. Interestingly, macropinocytosis can be stimulated in both pro- and anti-inflammatory macrophages in vitro although constitutive macropinocytosis is increased only in anti-inflammatory macrophages.

In vitro experiments using scanning electron microscopy, time-lapse imaging, and confocal microscopy demonstrated that macrophages form membrane ruffles in cell culture media that may circularize and close, leading to macropinosome formation and macropinocytosis. It was, however, unknown whether macrophages are able to form membrane protrusions in the extracellular matrix against proteoglycans, collagen and other fibrous proteins and despite increased arterial stiffness observed in atherosclerotic arteries. Using high-resolution TEM imaging and 3D reconstruction of macrophage foam cells, the data presented herein identified macrophages in the subendothelial layer of human and murine atherosclerotic arteries that demonstrate the full cycle of macropinocytosis, including single membrane protrusions, curved ruffles, macropinocytotic cups and formation of intracellular vesicles resembling macropinosomes. Immunoelectron microscopy localized LDL lipoproteins in cup-like structures and closed macropinosomes suggested that macrophage lipid macropinocytosis occurred in human atherosclerotic arteries. Quantification of DiI-LDL uptake by arterial macrophages in the absence and presence of EIPA were consistent with these results and suggested lipid internalization by macrophages via macropinocytosis in atherosclerotic lesions in vivo. Furthermore, ultrastructural analysis of atherosclerotic intima did not identify solid particles, microbes or fragments of apoptotic cells near plasma membrane protrusions, indicating phagocytosis- and efferocytosis-independent stimulation of plasma membrane activities in macrophages. It is also important to add that agg-LDL-induced phagocytotic cups were expected to be significantly smaller [50-300 nm] than the observed parallel membrane protrusions (1 μm in diameter or larger).

In a large cohort of patients hospitalized with atherosclerotic coronary artery disease (ST- and non-ST-segment elevation myocardial infarction and unstable angina), approximately half had admission levels of LDL lower than 100 mg/dl and almost 20% of patients had LDL<70 mg/dl. Although these results indicate that the therapeutic goal of circulating LDL levels needs to be even lower, this may not be feasible due to potential toxicity and increased rate of adverse effects associated with more aggressive lipid-lowering therapies and their limitations to further lower LDL levels. Previous studies demonstrated that macrophage foam cell formation can be induced when macropinocytosis is stimulated in the presence of 5 mg/dl nLDL in vitro. In human intima, the cholesterol level ranges from 16 to 251 mg/dl (mean concentration: 102±5 mg/dl). These results suggest that if inflammatory cytokines, growth factors and matrix proteins that stimulate macrophage macropinocytosis are upregulated in the arterial wall, foam cell formation may occur even in patients with normal or low LDL levels. Taken together, these results suggest the need for a combinatorial therapy that lowers circulating LDL levels, improves the barrier function of endothelial cells to attenuate LDL uptake into the arterial wall and inhibits macrophage internalization of subendothelially retained lipids.

In one embodiment the disclosed inhibitory agent inhibits LDL macropinocytosis in macrophages to reduce the generation of macrophage foam cells. In an additional embodiment the disclosed inhibitory agent induces, promotes, or enhances the reduction of atherosclerotic plaques in a subject with atherosclerotic vascular disease. In another embodiment the disclosed inhibitory agent prevents the formation of atherosclerotic plaques in a subject in need thereof.

Although imipramine's use may be limited due to its side effects in the central nervous system, the disclosed invention demonstrates the efficacy of repurposed macropinocytosis inhibitors for pharmacological treatment of atherosclerosis.

Excessive uptake of LDL by macrophages in the arterial wall is a critical process in the development and progression of atherosclerosis. The precise mechanisms, however, that mediate macrophage internalization of lipids, leading to foam cell formation, initiation and development of atherosclerotic lesions remain incompletely understood. The present invention provides the first set of compelling examples supporting an important role for macrophage macropinocytosis of LDL in atherosclerotic arteries in vivo, thus challenging the decades-old dogma of SR-mediated mechanisms of modified LDL uptake as the only mechanism mediating macrophage foam cell formation and atherosclerosis development. Although lipid lowering therapy is the mainstay for the treatment of atherosclerotic vascular disease and prevention of its cardiovascular consequences, considering additional therapeutic strategies targeting LDL transport across the dysfunctional endothelial layer, macrophage macropinocytosis of subendothelially retained lipids and macrophage reverse cholesterol transport seem important. For example, pharmacological inhibitors of stimulated macrophage macropinocytosis that have no effect on constitutive fluid-phase uptake of antigens by dendritic cells may afford synergistic outcomes with traditional lipid lowering therapies and provide more efficacious strategies for the treatment atherosclerotic vascular disease. Another embodiment provides a method for treating atherosclerotic vascular disease in a subject in need thereof by administering to the subject the combination of effective amount of a pharmaceutical composition including LDL lowering therapies and inhibitors of macrophage macropinocytosis to lower circulating LDL levels, improve the barrier function of endothelial cells to attenuate LDL uptake into the arterial wall and inhibit macrophage internalization of LDL for the treatment atherosclerotic vascular disease.

1. Imipramine

Pharmacological blockade of Na+-H+ exchanger 1 (NHE1) using 5-(N-ethyl-N-isopropyl)amiloride (EIPA) is currently regarded as the most effective and selective approach to inhibit macropinocytosis both in vitro and in vivo. However, possible effects of these drugs on transmembrane Na+ transport, pH regulation and water homeostasis, in addition to their capacity to block macropinocytosis, limit their use as therapeutic agents in cardiovascular medicine. Therefore, it is important to utilize a clinically relevant inhibitor of macrophage macropinocytosis that lacks regulatory effects on NHE1 and test its ability to inhibit atherosclerosis development. The inventors performed a large unbiased-screen of an FDA-approved drug library and identified a potent (IC50=131 nM), non-toxic [selectivity index (CC50/IC50)>300] low MW compound (imipramine) that selectively inhibits macropinocytosis in macrophages, independent of NHE1 regulation.

Imipramine is a tricyclic antidepressant (TCA) with high oral bio-availability (95%) and a half-life of nearly 20 hours that is clinically used in children to treat enuresis and adults with depression. In one embodiment, the disclosed inhibitory agent inhibits LDL macropinocytosis in macrophages. In a preferred embodiment the inhibitory agent is imipramine or any additional macropinocytosis inhibitors independent of their effects on NHE1 activity.

B. Pharmaceutical Compositions

Pharmaceutical compositions including macrophage macropinocytosis inhibitors are provided. Pharmaceutical compositions containing the compounds can be formulated for enteral or parenteral (intramuscular, intraperitoneal, intravenous (IV) or subcutaneous injection, intravitreal) routes of administration, topical administration, or using bioerodible inserts and can be formulated in dosage forms appropriate for each route of administration.

In some embodiments, the amount of macrophage macropinocytosis inhibitor in the formulation can be from 1.0 mg/mL to 100 mg/mL, about 1.0 mg/mL to about 50 mg/mL protein, about 5.0 mg/mL to about 25 mg/mL. In another embodiment, the amount of macrophage macropinocytosis inhibitor in the formulation can be about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. The composition can be formulated to achieve a concentration of about 9 mg/mL.

1. Formulations for Parenteral Administration

In some embodiments, the disclosed macrophage macropinocytosis inhibitor compositions are administered in an aqueous solution, by parenteral injection, typically by oral administration. The formulation may also be in the form of a suspension or emulsion. In general, pharmaceutical compositions are provided including effective amounts of macrophage macropinocytosis inhibitor, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

In some embodiments, the macrophage macropinocytosis inhibitor is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before all of the first agent is released. In still other embodiments, both agents are released at the same time over the same period of time or over different periods of time.

In one embodiment, the extended release composition includes microparticles having a diameter from about 1 μm to about 10 μm. The microparticles can have a diameter of 1 μm, 2 μm, 3 μm, 4 μm, 5 μm, 6 μm, 7 μm, 8 μm, 9 μm, or 10 μm. In another embodiment, the extended release compositions include nanoparticles have a diameter ranging from 10 nm to 950 nm. The nanoparticles can have a diameter of 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 950 nm.

In some embodiments, the macrophage macropinocytosis inhibitor composition releases the macrophage macropinocytosis inhibitor at a steady rate for 30, 60, 90, or 120 days after administration.

2. Oral Immediate Release Formulations

Some embodiments provide formulations for oral administration. Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER© 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

3. Controlled Delivery Polymeric Matrices

The compositions disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the disclosed compositions, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery.

C. Dosing Regimen

In some in vivo approaches, the macrophage macropinocytosis inhibitor compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed macrophage macropinocytosis inhibitor compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed macrophage macropinocytosis inhibitor compositions, generally dosage levels of 0.001 to 20 mg/kg of body weight daily are administered to mammals.

D. Subjects to be Treated

Macrophage foam cell formation is an important process in atherosclerotic plaque development. Uptake and storage of plasma lipoprotein-derived cholesterol within monocyte-derived macrophages affects macrophage functions in ways that influence plaque development and stability. The disclosed methods and compositions can be used to treat or reduce the symptoms of diseases that result from atherosclerotic vascular disease.

In some embodiments, atherosclerotic vascular disease is characterized by high levels of circulating LDL relative to subjects without the disease or condition. In some embodiments, atherosclerotic vascular disease is characterized by increased macrophage macropinocytosis of LDL. In additional embodiments, atherosclerotic vascular disease is characterized by increased formation of macrophage foam cells. In another embodiment, atherosclerotic vascular disease is characterized by the formation of atherosclerotic plaques in the arterial wall. One embodiment provides a method for treating atherosclerotic vascular disease in a subject in need thereof by administering to the subject an effective amount of a pharmaceutical composition including inhibitors of macrophage macropinocytosis.

Another embodiment provides a method for treating atherosclerotic vascular disease in a subject in need thereof by administering to the subject the combination of effective amount of a pharmaceutical composition including LDL lowering therapies and inhibitors of macrophage macropinocytosis to lower circulating LDL levels, improve the barrier function of endothelial cells to attenuate LDL uptake into the arterial wall and inhibit macrophage internalization of LDL for the treatment atherosclerotic vascular disease. Representative medicines that can be used to lower LDL levels in the subject include, but are not limited to, statins such as atorvastatin, fluvastatin, lovastatin, pravastatin, rosuvastatin, simvastatin, pitavastatin, and combinations thereof.

### EXAMPLES

Materials and Methods:

Reagents

Nile Red, 5-(N-Ethyl-N-isopropyl)amiloride (EIPA), imipramine, Oil Red O, SOD, catalase, CuSO4, PMA and FITC-Dextran were purchased from Sigma-Aldrich (St. Louis, Mo., USA). EIPA and imipramine solutions were prepared in DMSO and water, respectively. Mouse recombinant granulocyte-macrophage colony-stimulating factor (GM-CSF), interleukin-4 (IL-4), human recombinant TNFα and PDGF were obtained from Peprotech (Rocky Hill, N.J., USA). Human nLDL, ox-LDL, ac-LDL and Dil-nLDL were obtained from Kalen Biomedical, LLC (Montgomery Village, Md., USA). M-CSF, Neonatal Heart Dissociation Kit, anti-CD11b microbeads, CD36-APC antibody, SR-A-APC antibody and IgG-APC antibody were purchased from Miltenyi Biotec Inc. (San Diego, Calif., USA). AD-GFP and AD-HRasG12V adenoviruses were kindly provided by Dr. Brian Stansfield (Augusta University, Augusta, Ga., USA). AAV8-PCSK9 was obtained from Vigene Biosciences. FITC-transferrin, Alexa Fluor 488-albumin, Alexa Fluor 488-Dextran, Vybrant® CFDA Cell Tracer Kit, Amplex™ Red Cholesterol Assay Kit, FM™ 4-64 Dye and Cell Tracker™ Deep Red Dye were obtain from ThermoFisher (Rockford, Ill., USA). TaqMan® Reverse Transcriptase kit and SYBR Green Super mix were obtained from Applied Biosystems (Carlsbad, USA). Anti-F4/80-APC (clone BM8) and IgG control antibodies were obtained from Biolegend (San Diego, Calif., USA). Western diet were obtained from Envigo (Indianapolis, Ind., USA).

Cell Culture

Human aortic endothelial cells (HAECs) were obtained from Lonza (Houston, Tex.). THP-1 monocytes were purchased from Sigma (St. Louis, Mo.). Cells were cultured in humidified 5% CO2 at 37° C. using appropriate culture medium. HAECs were cultured in EBM-2 MV medium supplemented with EGM-2 MV kit (Lonza). Mouse bone marrow-derived monocytes and THP-1 monocytes were cultured in RPMI-1640 medium containing 10% FBS, 100 IU/mL of penicillin G and 100 μg/mL streptomycin. Bone marrow-derived monocytes were differentiated into macrophages using murine M-CSF (20 ng/ml, 6 days) as previously described (64). THP-1 monocytes were maintained in cell-free conditioned media and differentiated into macrophages using human M-CSF (20 ng/ml, 9 days). Cells were cultured in the absence of M-CSF for at least 24 hr prior to experiments. RAW 264.7 macrophages were cultured in DMEM medium containing 10% FBS, 100 IU/mL of penicillin G and 100 μg/mL streptomycin.

Animals

Mice were housed in accordance to the National Institutes of Health (NIH) guidelines in an AAALAC-accredited experimental animal facility under controlled environment. All mouse studies were approved by the Institutional Animal Care and Use Committee at Augusta University. C57BL/6J (stock #000664), LysMCre (stock #019096), CD36−/− (stock #019006), SR-A−/− (stock #006096) and ApoE−/− (stock #002052) mice were purchased from The Jackson Laboratory (Bar Harbor, Me., USA). NHE1f/f mice were kindly provided by Dr. Dandan Sun (University of Pittsburgh, Department of Neurology). LysMCre+NHE1f/f (NHE1ΔM) mice were generated by crossing NHE1f/f mice with LysMCre transgenic mice. Littermate LysMCre-NHE1f/f (NHE1f/f) mice were used as controls. CD36−/−SR-A−/− mice were generated by breeding CD36−/− mice with SR-A−/− mice. All mice were genotyped by PCR amplification of tail DNA. Every effort was made to minimize animal suffering and reduce the number of animals used. All mice were fed with standard chow diet until a week before the surgery and/or Western diet at the age of 8-10 weeks.

Mice were given a single injection of AAV-PCSK9 (1×1011 VG) and fed a Western diet (Envigo, TD.88137, Indianapolis, Ind.). One week after AAV-PCSK9 injection, mice underwent partial carotid ligation surgery as previously described (67) and Western diet was continued for another 3 weeks. In separate experiments, mice were given a single injection of AAV-PCSK9 (1×1011 VG) and fed a Western diet for 16 weeks. Mice were anesthetized (isoflurane inhalation, 3%) and exsanguinated. Blood was collected from left ventricle using a heparinized syringe fitted with a 21 G needle. Aortic arch and carotid arteries were collected, and gross macroscopic images were acquired. For molecular histology studies, carotids and aortic arch were fixed in 4% paraformaldehyde (PFA), embedded in OCT compound and stored at −80° C. until use.

Human Atherosclerotic Arteries

Human atherosclerotic arteries were obtained from cadavers by Dr. Joseph White (Department of Pathology, Augusta University). The heart and aortic tissue were isolated within 24 hr of death. The isolated tissues were thoroughly rinsed with ice-cold phosphate-buffered saline (PBS), cleaned of perivascular fat and fixed in EM fixative (2% Glutaraldehyde in 0.1 M Sodium Cacodylate Buffer, pH 7.4) for TEM analysis. The use of human cadaveric specimens was approved by the Institutional Biosafety Committee at Augusta University.

Isolation of Marine Macrophages

Collection of thioglycollate-elicited peritoneal macrophages: Thioglycollate medium (3%) was injected into the peritoneum of 8-week-old male LysMCre-NHE1f/f and LysMCre+NHE1f/f mice. Four days later, 100 μg of Dil-LDL was injected intraperitoneally. After 24 hours, mice were anesthetized (isoflurane inhalation, 3%) and sacrificed by cervical dislocation. Peritoneal cells were harvested by lavage with approximately 10 ml sterile ice-cold PBS. Cells were sedimented by centrifugation at 300 g for 5 min and seeded in tissue culture plates. After 2-3 hours, the plates were washed to remove non-adherent cells. Macrophage Dil-nLDL uptake was quantified using FACS analysis (Ex: 549 nm, Em: 565 nm).

Generation of mouse bone marrow-derived macrophages: Femur and tibia of sacrificed mice were cleaned of adherent muscle and connective soft tissue. Bone marrow containing cavities were exposed by cutting both ends of bones, and a 25-gauge needle syringe containing Harvest Buffer (HBSS, containing 10 mM Hepes, 4 mM sodium bicarbonate and 4% heat-inactivated FBS) was used to flush the bones and bone marrow cells were collected. Bone marrow monocytes were cultured in RPMI-1640 medium and differentiated into macrophages using 20 ng/ml M-CSF for 6 days.

Isolation of macrophages from atherosclerotic arteries: 8-10-week-old ApoE^{-/-} mice were fed a Western diet for 12 weeks. Mice were treated with vehicle or EIPA (25 mg/kg/day, i.p.) for 3 days. On the day of sacrifice, mice were retro-orbitally injected with vehicle or 100 μg Dil-nLDL. After 4 hours, mice were anaesthetized and perfused with ice-cold PBS for 5 min and the entire thoracic aorta with LCA was isolated. Following removal of the adventitia, macrophages were isolated using the Neonatal Heart Dissociation Kit (Miltenyi Biotech.) and CD11b microbeads (Miltenyi Biotech.). Cells were stained with anti-F4/80-APC (clone BM8) or IgG control antibodies (Biolegend). Dil fluorescence was measured in F4/80-positive macrophages [Dil (Ex: 549 nm, Em: 565 nm) and APC (Ex: 640 nm, Em: 670 nm)].

M-CSF and PDGF Expression in Human Atherosclerotic and Control Arteries

To examine M-CSF and PDGF mRNA expression in human non-atherosclerotic and atherosclerotic arteries, the publicly available independent human atherosclerosis cohorts with gene expression data were downloaded from the Gene Expression Omnibus (GEO). The accession number was GSE43292. Gene expression profiling was generated using the Affymetrix Human Gene 1.0 ST Array. CELL files were downloaded from GEO and then analyzed using R/BioConductor package with RMA method and custom PERL scripts.

Monocyte Adhesion Assay

Adhesion studies were performed using the human monocyte cell line THP-1. Vehicle- or EIPA (25 μM, 24 hours)-treated THP-1 monocytes were labeled using Vybrant™ CFDA SE Cell Tracer Kit (ThermoFisher). Human aortic endothelial cell monolayers grown on coverslips in a 24-well plate were treated with vehicle (PBS) or TNFα (10 ng/ml) for 4 hours. Endothelial cells were labeled with Cell Tracker™ Deep Red Dye (ThermoFisher). Fifty microliter of THP-1 monocyte suspension containing 1×106 cells was added to each well of endothelial cells, and the plates were gently agitated and placed in a cell culture incubator for 1 hour. At the end of the incubation period, the monolayer was gently washed three times with cold PBS. After the final wash, coverslips were stained with Hoechst 33342 (ThermoFisher) and mounted onto slides using mounting medium. Fluorescent signals were detected using a Zeiss 780 upright confocal microscopy. Quantification was completed with Image-Pro Plus software (Media Cybemetics, Bethesda, Md., USA).

Measurement of Cholesterol Efflux

RAW 264.7 macrophages were pretreated with vehicle or EIPA (25 μM, 1 hour) and incubated with PMA (1 μM) in the presence or absence of Dil-LDL (50 μg/mL) at 37° C. for 24 h. Cells were washed 3 times with PBS, RPMI-1640 medium containing 1% FBS was added and cells incubated for another 24 hours. Finally, the medium was collected and Dil fluorescence intensity in the media was measured using a CLARIOstar multi-mode microplate reader.

Cytokine Secretion

Wild type BMDM were incubated with vehicle or treated with EIPA (25 μM, 24 hours). Cytokine secretion into the media was quantified using the LEGENDplex™ (mouse inflammation panel) bead-based immunoassay according to the manufacturer's instructions (BioLegend, San Diego, Calif., USA). Data were acquired using four-color BD FACSCalibur (BD Biosciences, San Jose, Calif., USA) and analyzed using the LEGENDplex Data Analysis software (BioLegend).

Lesion Quantification and Morphological Analysis Atherosclerotic Vessels

ORO staining: Atherosclerosis development in carotid arteries and whole aorta were studied initially by gross imaging followed by Oil Red O (ORO) staining and plaque quantification. Briefly, the entire aorta was isolated, cleaned of peri-adventitial fat, fixed and imaged on the surface of black silicone-coated dissection dishes. Left carotid arteries were embedded in OCT and 10 μm thick cross-sections were prepared. The arteries were divided into 3 regions based on their distance from the aortic arch: proximal (0-400 μm), middle (2,000-2,400 μm) and distal (4,000-4,400 μm). Frozen sections and whole aortas were then stained with ORO solution (2%) and images were taken using light microscope equipped with a camera under 20× and 1.6× objective lens. The total area, lumen area and ORO positive area were measured for six sections/anatomical position per mouse using Image J software. The ratio of ORO positive area to arterial area is represented as plaque area. Plasma total cholesterol concentrations were determined by the Amplex™ Red Cholesterol Assay Kit (ThermoFisher, Rockford, Ill.). Plasma lipid profiles were obtained by column fractionation and measurements of fraction cholesterol content.

Hematoxylin and Eosin (H&E) or Masson's trichrome staining: Frozen sections were also used for Hematoxylin and Eosin (H&E) or Masson's trichrome staining following standard procedures to evaluate the lesion size and collagen content, respectively.

Orcein and Martius Scarlet Blue (OMSB) staining: OMSB was used for qualitative and quantitative analysis of atherosclerotic plaques as previously described. Briefly, left carotid arteries were dissected out, fixed in 4% buffered paraformaldehyde (pH=7.4) for at least 48 hr, and then routinely processed and embedded in paraffin (tissue processor TP1020; Leica, Wetzlar, Germany). Cross sections were cut every 5 m along the artery (starting from the proximal end) and mounted serially on silanized slides. Sections were stained and mounted automatically using Leica ST5010 Autostainer XL combined with Leica CV5030 Glass Coverslip. Deparaffinized sections through xylene and ethanol, and rehydrate to water. Postfix sections in Bouin's fixative at 56° C. for 1 hour, then rinse for 10 min in tap water to remove traces of picric acid. Stain with orcein for 20 min at 56° C. Differentiate in acidic alcohol for 2 min and then transfer to 96% ethanol for 30 s. Stain with martius yellow for 3 min and rinse in distilled water (2×2 min). Stain with crystal scarlet for 10 min. Differentiate with phosphotungstic acid 2 min and transfer to distilled water (for about 10 s). Stain with methyl blue 5 min. Rinse in acetic acid water 3 min, dehydrate through ethanol and carbol-xylene, clear in xylene, and mount in DPX or other resin. Staining of collagen with picrosirius red. The sections were examined and digital images of stained arteries were recorded using Olympus dotSlide system (Olympus, Tokyo, Japan). Segmentation algorithm allowed to distinguish vessel wall area, lumen area, plaque area, and area of the plaque occupied by collagen. The results were presented as areas of identified components in μm2. Further morphological parameters were calculated: relative inner vessel area [IVA %=plaque area/(plaque area+lumen area)×100%], relative collagen area (collagen %=collagen area/plaque area×100%).

Flow Cytometry

Flow cytometry experiments were performed using the BD Accuri C6 flow cytometer using a standard protocol.

Foam cell formation: THP-1 macrophages or bone marrow-derived macrophages were treated with vehicle, known macropinocytosis stimulators (PMA 1 μM, M-CSF 100 ng/mL or PDGF 200 ng/mL)+/− macropinocytosis inhibitors (imipramine 10 μM or EIPA 25 μM; 1 hour pre-incubation) in the presence of nLDL, ox-LDL, or ac-LDL (50 g/mL) for 24 hours. Cells were fixed in 2% PFA and stained with Nile Red (50 ng/mL) for 7 min. Fluorescence intensity was measured using the FL3 channel for Nile Red (Ex: 488 nm, Em: 670 nm).

Clathrin- and caveolin-mediated endocytosis: Receptor-mediated uptake of FITC-transferrin (1 μg/mL; clathrin-mediated endocytosis) and Alexa Fluor 488-albumin (1 g/mL; caveolin-mediated endocytosis) was investigated in RAW 264.7 macrophages following their incubation with ±EIPA (25 μM; 1 hour pre-incubation). Fluorescence intensity was measured using the FL1 channel for FITC and Alexa Fluor 488 (Ex: 488 nm, Em: 530 nm).

Cell viability assay: RAW 264.7 macrophages were plated in 24 well plates at a density of 1×106 cells/mL. Twenty four hours later, macrophages were pre-incubated with vehicle or EIPA (25 μM, 1 hr) and treated with PMA (1 μM, 4 hrs). Cells were fixed in 2% PFA, resuspended in FACS buffer (2% BSA and 0.01% sodium azide in PBS), and stained with the LIVE/DEAD™ Fixable Far Red dye to identify live cells. Fluorescence intensity was measured using the FL4 channel (Ex: 650 nm, Em: 660 nm).

Agarose Gel Electrophoresis

THP-1 macrophages were treated±M-CSF (100 ng/mL) or ±PDGF (200 ng/mL) in the absence or presence of nLDL (50 μg/mL) for 24 hours. Cell culture medium was collected and sent to Kalen Biomedical, LLC for agarose gel electrophoresis at 4° C. Native LDL and ox-LDL directly obtained from Kalen Biomedical, LLC were used as negative and positive controls, respectively. nLDL (50 μg/mL) was incubated with 50 μM CuSO4 for 72 hours also served as a positive control.

Measurements of Plasma Total Cholesterol Levels

Total cholesterol levels were measured using the Amplex Red Cholesterol Assay Kit (ThermoFisher) according to the manufacturer's instructions.

Imaging Studies

Immunohistochemistry: Left carotid artery frozen sections were washed in PBS for 5 min, blocked, incubated with primary antibodies against CD68 (1:60, Invitrogen, overnight at 4° C.) and developed with reagents provided in the kit (Lab Vision™ Ultra Vision™ LP Detection System, ThermoFisher) according to manufacturer's instructions. Sections were counterstained with hematoxylin and mounted with cytoseal XYL medium. Images were captured using a phase-contrast microscope. CD68 positive area was calculated using Image Pro-Plus software (Media Cybernetics).

Scanning electron microscopy (SEM): Macrophages were fixed (4% PFA, 2% glutaraldehyde in 0.1 M sodium cacodylate solution) overnight at 4° C. Then, cells were dehydrated through a graded ethanol series (25%-100%) and washed with 100% ethanol before critical point drying (Tousimis Samdri-790, Rockville, Md.). Coverslips were mounted onto aluminum stubs and sputter coated with 3.5 nm of gold/palladium (Anatek USA-Hummer, Union City, Calif.). Cells were imaged at 20 KV using a Philips XL30 scanning electron microscope (FEI, Hillsboro, Oreg.). Number of membrane ruffles in AD GFP- and AD HrasG12V-overexpressing macrophages were normalized to total cell number in the microscopic field evaluated.

Transmission electron microscopy (TEM): Atherosclerotic ApoE−/− mouse and human aortic tissue were fixed in 4% paraformaldehyde, 2% glutaraldehyde in 0.1 M sodium cacodylate (NaCac) buffer, and pH 7.4, post fixed in 2% osmium tetroxide in NaCac, stained en bloc with 2% uranyl acetate, dehydrated with a graded ethanol series and embedded in Epon-Araldite resin. Sixty nm sections were cut with a diamond knife on a Leica EM UC6 ultra microtome (Leica Microsystems, Inc, Bannockburn, Ill.), collected on copper grids and stained with uranyl acetate and lead citrate. Tissue was observed in a JEM 1230 transmission electron microscope (JEOL USA Inc., Peabody, Mass.) at 110 kV and imaged with an UltraScan 4000 CCD camera & First Light Digital Camera Controller (Gatan Inc., Pleasanton, Calif.). Image stacks were analyzed using the Autotrace feature in Reconstruct software.

Immune electron microscopy (IEM): Using Immunostaining EM Grids with Nanogold and Silver Enhancement, human LAD were fixed in 4% paraformaldehyde and processed through a graded series of ethanol (25%, 50%, 70%, 80%, and 95%). Tissue was embedded in LR White Resin (Electron Microscopy Sciences, Ft. Washington, Pa.). Sections 75 nm in thickness were collected on 200 mesh nickel grids. Grids were baked in 60° C. oven for 5 min to seal sections to the grid. Grids, section-side down were floated on drops of etching solution (5% Sodium Metaperiodate in PBS) for 5 min and washed with PBS. Aldehydes were quenched 30 min with 1 M ammonium chloride in PBS. Grids were then blocked in Aurion Blocking Solution (Electron Microscopy Sciences, Ft. Washington, Pa.), in PBS for 2-4 hours at room temperature. Grids were floated on drops of primary antibody diluted in Aurion BSA-c buffer (Electron Microscopy Sciences, Ft. Washington, Pa.) in PBS overnight at 4° C. and washed in PBS. For single antibody labeling, grids were floated on drops of anti-primary species-specific Nanogold (Nanoprobes, Yaphank, N.Y.) reagent diluted (1:1000) in Aurion BSA-c buffer for 2 hours at RT. Nanogold particles were silver enhanced for 4 min using HQ Silver™ (Nanoprobes, Yaphank, N.Y.), then washed in ice cold DI H₂O to halt enhancement. Grids were stained with 2% Uranyl Acetate and Lead Citrate to increase contrast. Tissue was observed in a JEM 1230 transmission electron microscope (JEOL USA Inc., Peabody, Mass.) at 110 kV and imaged with an UltraScan 4000 CCD camera & First Light Digital Camera Controller (Gatan Inc., Pleasanton, Calif.).

Confocal imaging: Macropinocytosis was stimulated with PMA (1 μM, 5 min) in the presence of Alexa Fluor 488-Dextran (100 μg/mL). The plasma membrane was stained with FM™ 4-64 (ThermoFisher). Live cell imaging was performed using a Zeiss 780 upright confocal microscope. In separate experiments, peritoneal macrophages were cultured in coverslips for 4 hours, cells were fixed with 4% PFA and stained with Hoechst 33342 and mounted onto slides using mounting medium. Fluorescent (DiI and Hoechst 33342) signals were detected using a Zeiss 780 upright confocal microscope.

Overexpression of AD-HRasG12V in Macrophages

HRasG12V mutant adenovirus (AD-HRasG12V) and control adenovirus (AD-GFP) were obtained from Dr. Brian Stansfield. Adenovirus multiplicity of infection (MOI) was calculated according to the OD260 of the adenovirus. Macrophages grown at 60-70% confluence were incubated in 500 μl basal medium containing Ad-GFP or Ad-HRasG12V adenovirus at 200 MOI (multiplicity of infection). After 4 hours, media was replaced with fresh complete tissue culture medium for a continuous culture. Cells were treated as indicated within 48 hours after viral transfection and collected for flow cytometry. Overexpression of adenovirus was confirmed by measuring GFP fluorescence. Fluorescence intensity was measured using the FL1 channel GFP (Ex: 488 nm, Em: 530 nm).

Quantitative PCR

Total RNA of bone marrow cells was extracted using an RNA extraction kit (IBI Scientific, Dubuque, Iowa) according to the manufacturer's protocol. RNA concentration and quality were determined by taking OD at 260 and 280 nm using a Nano Drop. Complementary DNA was generated utilizing the TaqManReverse Transcriptase kit (Applied Biosystems, Grand Island, N.Y.). Real-time PCR was carried out with SYBR Green Super mix (Applied Biosystems). All amplifications were performed in triplicate, and GAPDH was used as the internal control.

Blood Pressure Measurement

Non-invasive tail-cuff plethysmography was used to measure systolic blood pressure. Mice were placed into clear acrylic holders and strained with a built-in nose cone, then the acrylic holders were put on the platform of CODA High Throughput system preheated to 37° C. Tail cuffs were placed at the base of the unstrained tails of mice and connected to the CODA High Throughput system. To ensure a more robust estimation of systolic blood pressure (SBP), the interquartile mean of SBP measurements achieved through 30 measurement cycles every day was used. The average of at least nine readings, taken in the quiescent state, following acclimatization, was recorded per animal. Mice were trained for 3 continuous days, then systolic blood pressure was measured and averaged for another 3 continuous days.

NMR Analysis

Mice were scanned using the Bruker Minispec Live Mice Analyzer (model mq7.5, the “LF50”). The machine acquires RF signals generated by the hydrogen spins from soft tissues such as adipose and muscle, and uses the contrast in relaxation times of the hydrogen spins, or the amplitude, duration, and spatial distribution of these NMR signals from the different tissues to estimate composition. Animals were placed in a clear, plastic cylinder (50 mm diameter) and kept immobile by insertion of a tight fitting plunger into the cylinder. The tube was then put into the sample chamber of the instrument and measurements were recorded.

Statistical Analysis

All data are presented as mean±SEM. Data were analyzed by GraphPad InStat software (GraphPad Software, Inc.). Student's t-test was performed to analyze differences between two groups. Comparisons among multiple groups were made using one/two way ANOVA with an appropriate post hoc test (Tukey). A p<0.05 was considered to be statistically significant.

### Example 1. Pharmacological Inhibition of Macropinocytosis Decreases Atherosclerotic Lesion Development in Both Wild-Type and CD36^{-/-}/SR-A^{-/-} Mice

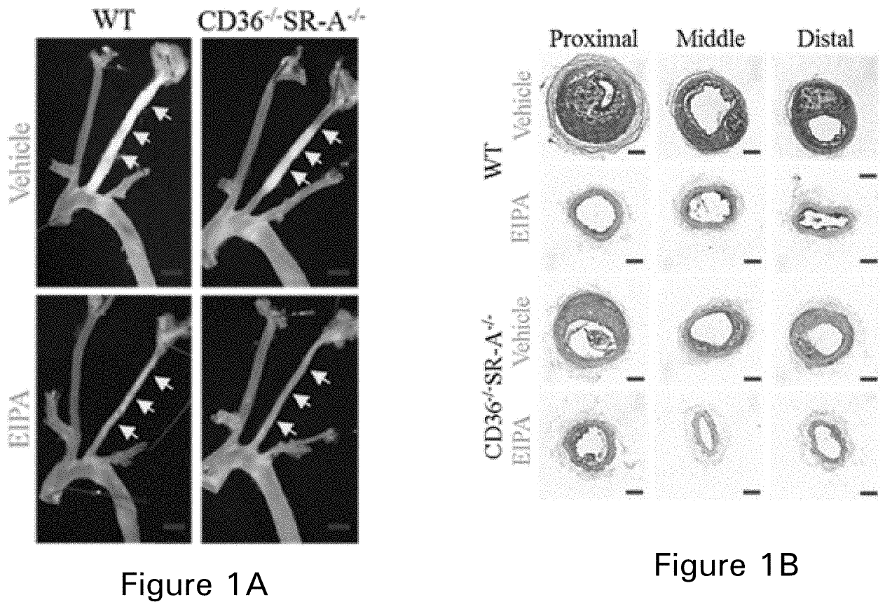

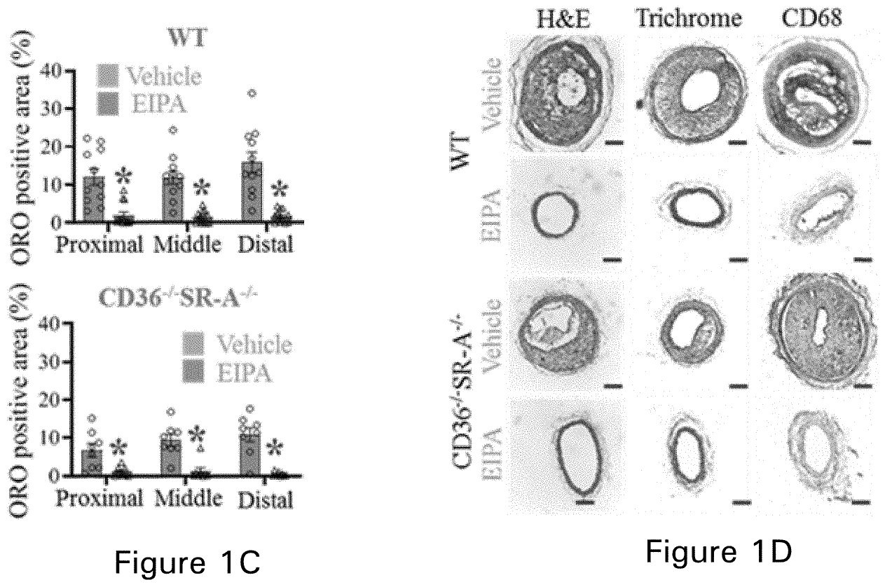

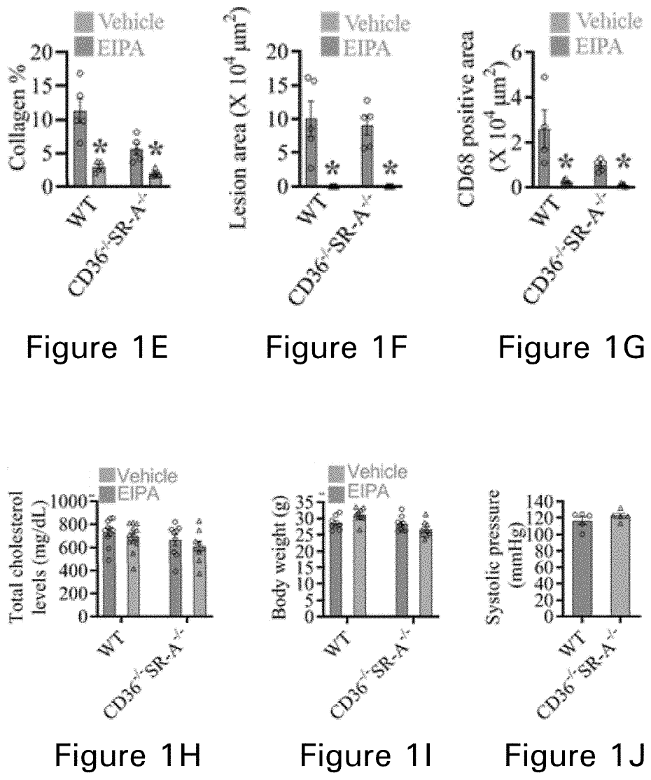

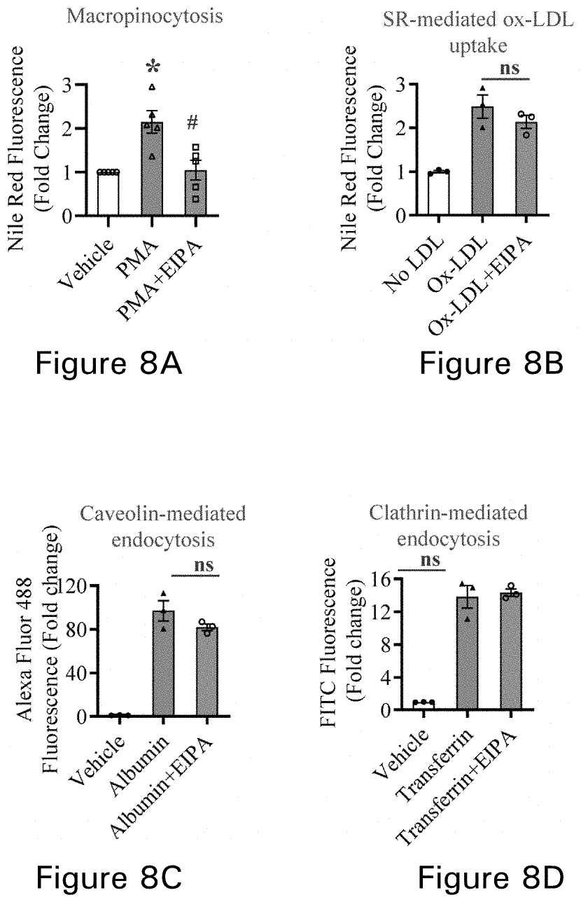

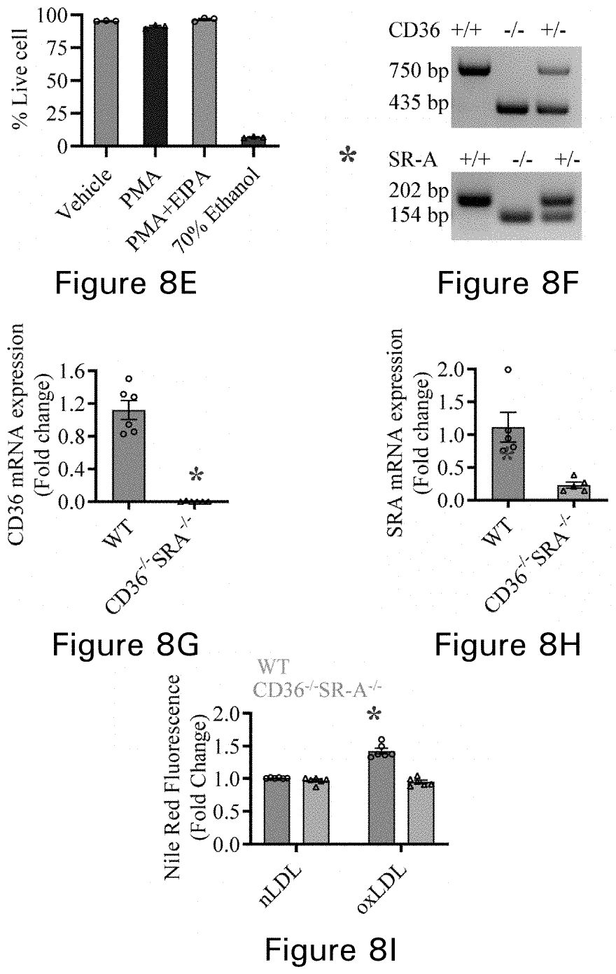

Macropinocytosis (a.k.a. fluid-phase endocytosis) is an endocytic mechanism mediating internalization of extracellular fluid and nonspecific bulk uptake of pericellular solutes. Pharmacological blockade of Na⁺—H⁺ exchanger 1 (NHE1) using 5-(N-ethyl-N-isopropyl)amiloride (EIPA) is currently regarded as the most effective and selective approach to inhibit macropinocytosis both in vitro and in vivo. To investigate the relative contribution of macrophage macropinocytosis versus SR-mediated pathways to atherosclerosis development, hypercholesterolemic C57BL 6 wild-type (WT) and CD36^{-/-}/SR-A^{-/-} mice (PCSK9-AAV-mediated LDLR knockdown) were treated with vehicle or EIPA using subcutaneously implanted osmotic pumps. Control experiments confirming selectivity demonstrate that EIPA blocks macrophage macropinocytosis without inhibiting other endocytic pathways, including SR-mediated ox-LDL uptake, clathrin-mediated transferrin endocytosis and caveolin-dependent albumin internalization (FIGS. 8A-D). Cytotoxicity assessment of EIPA using the LIVE/DEAD Fixable Cell Stain indicated no cell death in these experiments (FIG. 8E). Continuous infusion of EIPA substantially inhibited atherosclerotic lesion area in both WT (˜80%) and CD36^{-/-}/SR-A^{-/-} (˜80%) mice (FIGS. 1A-C, FIGS. 8F-H). Functional knockdown of ox-LDL uptake in CD36^{-/-}/SR-A^{-/-} macrophages is shown in FIG. 81. Vehicle-treated CD36^{-/-}/SR-A^{-/-} mice developed ˜32% smaller lesion area compared to WT controls (FIG. 1C), consistent with previous reports. Histological characterization of WT and CD36^{-/-}/SR-A^{-/-} atherosclerotic arteries using H&E, Masson's trichrome staining and CD68 immunolabeling are shown in FIG. 1D-G. Collagen content, atherosclerotic lesion area and CD68⁺ macrophage staining were significantly decreased in EIPA-treated mice compared with vehicle-treated WT and CD36^{-/-}/SR-A^{-/-} controls. Plasma cholesterol levels, body weight and blood

TABLE 1
Secretion of inflammatory cytokines by vehicle-
and EIPA-treated WT macrophages (n = 3).

| Treatment | TNFα (pg/mL) | MCP-1 (pg/mL) | IL-6 (pg/mL) |
|---|---|---|---|
| Vehicle | 7.83 ± 1.89 | 38.04 ± 6.03 | 2.40 ± 0.58 |
| EIPA | 4.76 ± 0.77 | 22.19 ± 2.30 | 1.82 ± 0.40 |
| P-values | 0.2054 | 0.0699 | 0.4614 |

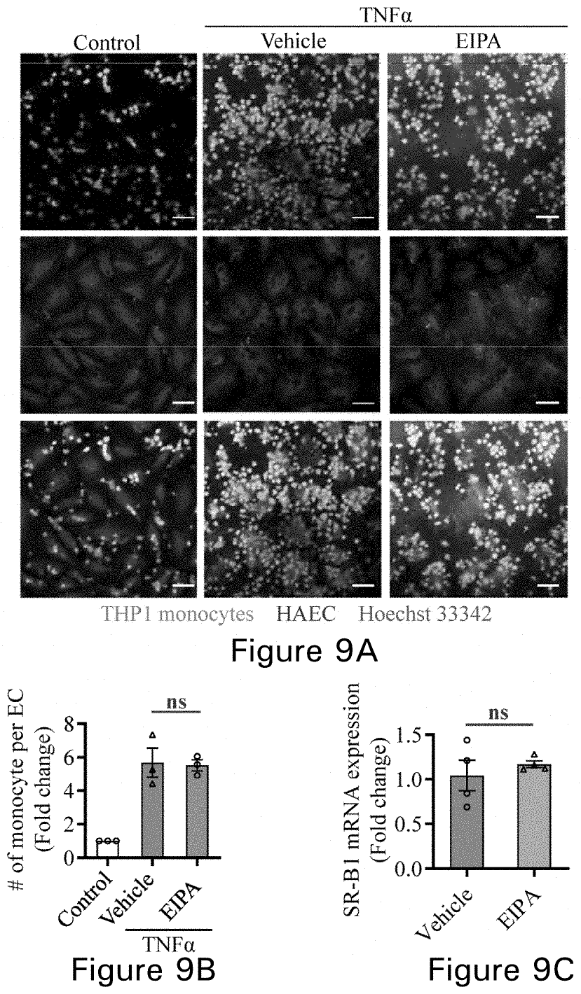

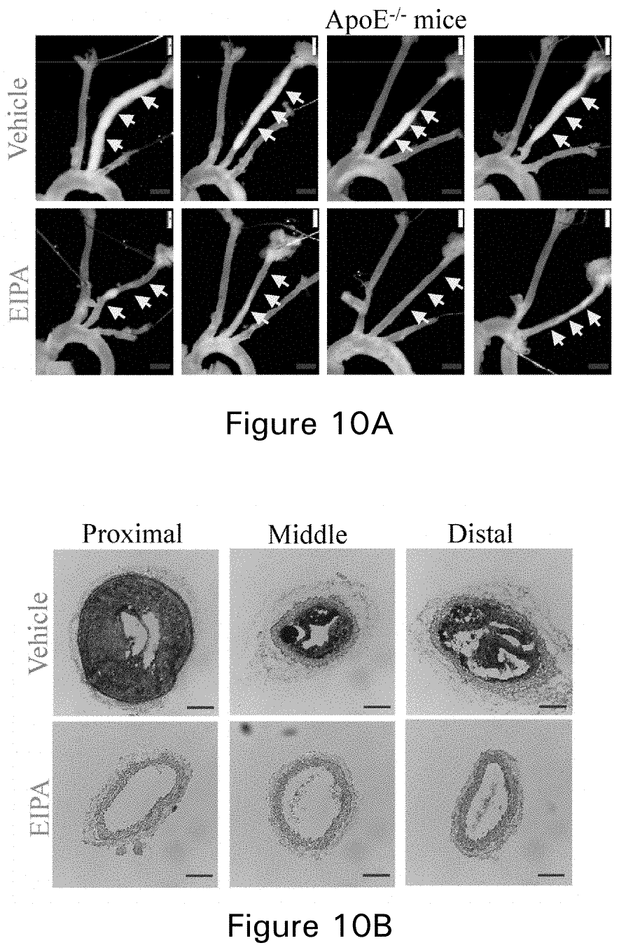

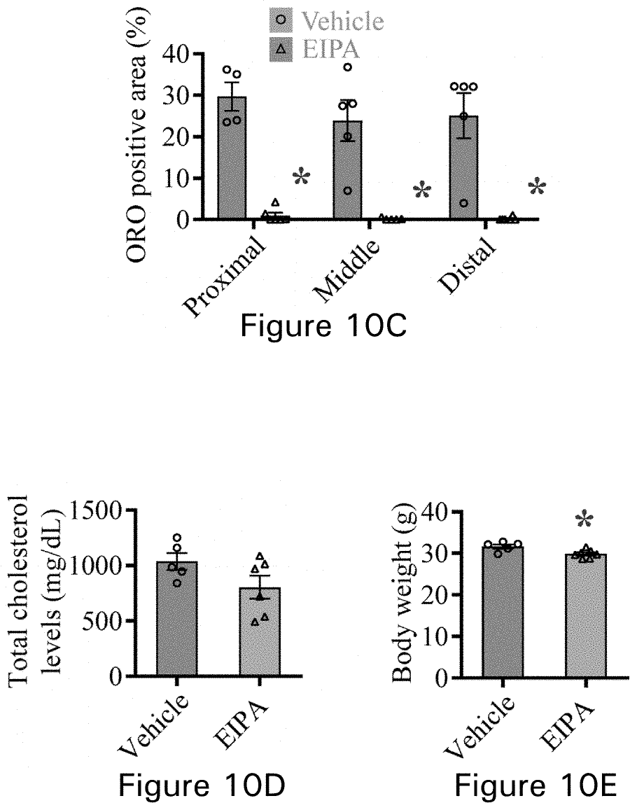

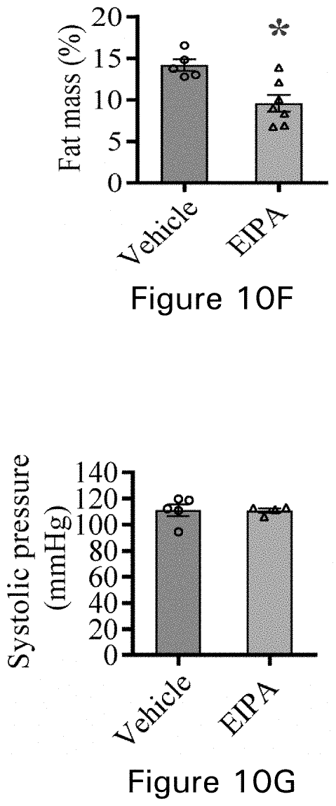

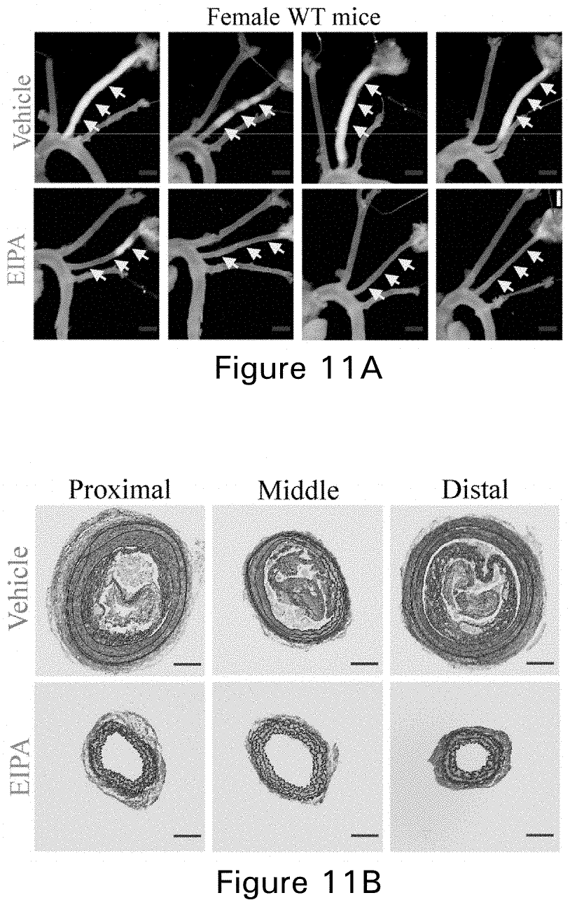

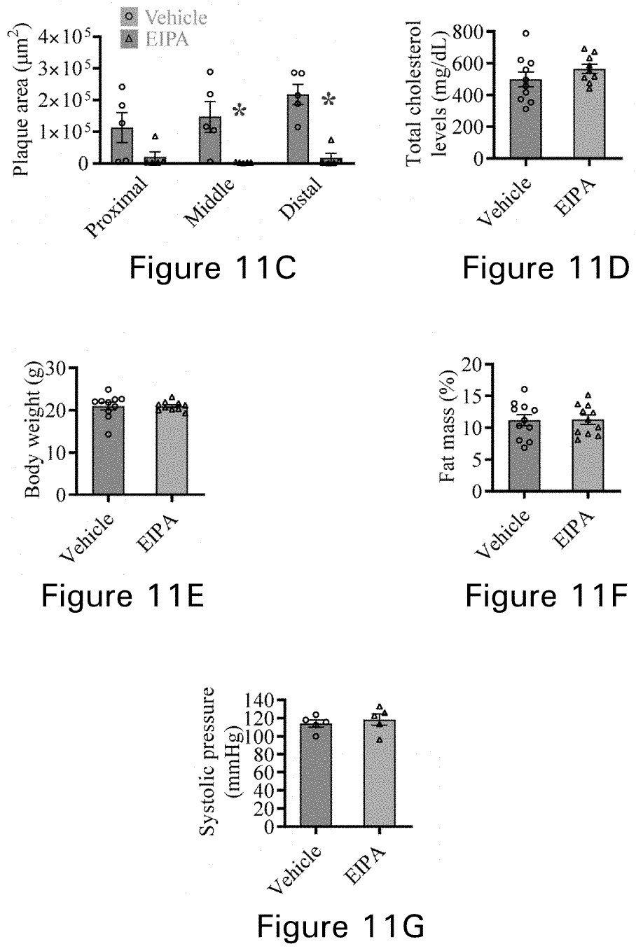

pressure were not different between experimental groups (FIGS. 1H-J). Next, EIPA's effect on macrophage secretion of inflammatory cytokines and monocyte adhesion to endothelial cells in the absence of exogenous lipids was investigated. Bead-based multiplex immunoassays demonstrated that EIPA does not inhibit macrophage secretion of TNF-α, MCP-1, and IL-6 (Table 1). In addition, results of confocal microscopy indicated that EIPA treatment does not inhibit monocyte adhesion to TNFα-treated endothelial cells in vitro (FIGS. 9A-9B). A recent study demonstrated that SR-B1 promotes endothelial cell LDL transcytosis and contributes to atherosclerosis. RT-PCR experiments indicated that EIPA does not increase SR-B1 mRNA levels in human aortic endothelial cells (FIG. 9C). As all mouse models of atherosclerosis have limitations, experiments were performed to confirm results obtained in male PCSK9-AAV-induced LDLR-downregulated mice (FIG. 1A-1C). As shown in FIGS. 10A-10G and FIG. 11A-G, pharmacological inhibition of macropinocytosis using EIPA inhibited atherosclerosis in Western diet-fed ApoE^{-/-} mice and in female LDLR-deficient animals.

### Example 2. Genetic Stimulation of Macropinocytosis Promotes Cholesterol Accumulation in WT and CD36−/−/SR-A−/− Macrophages, Leading to Foam Cell Formation

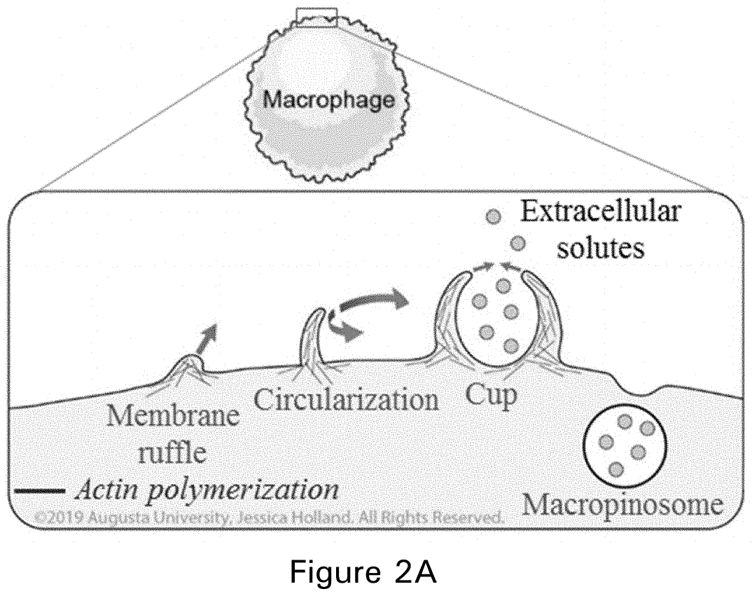

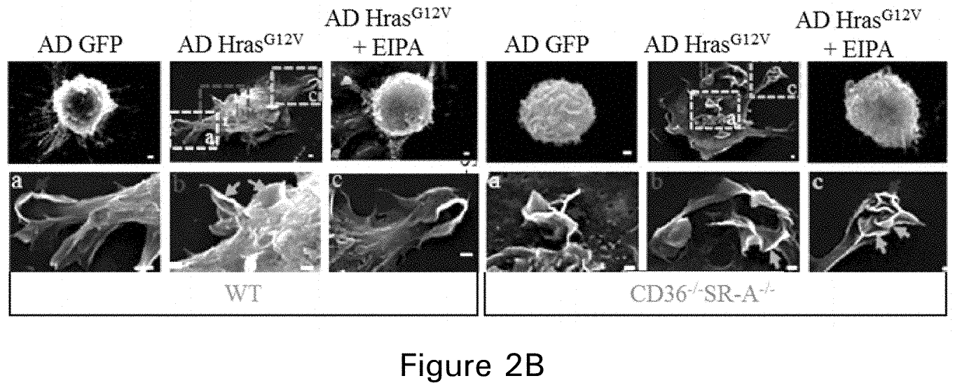

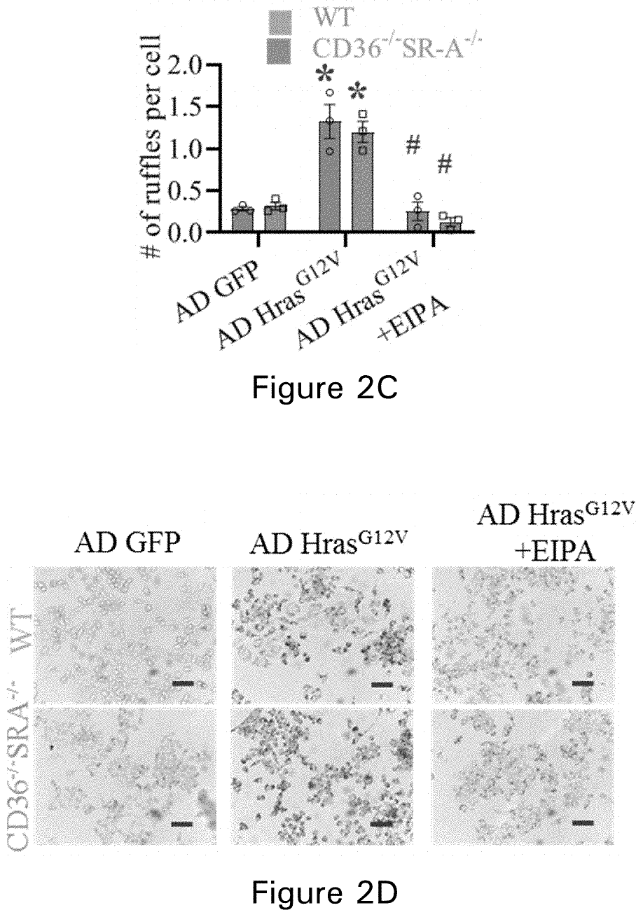

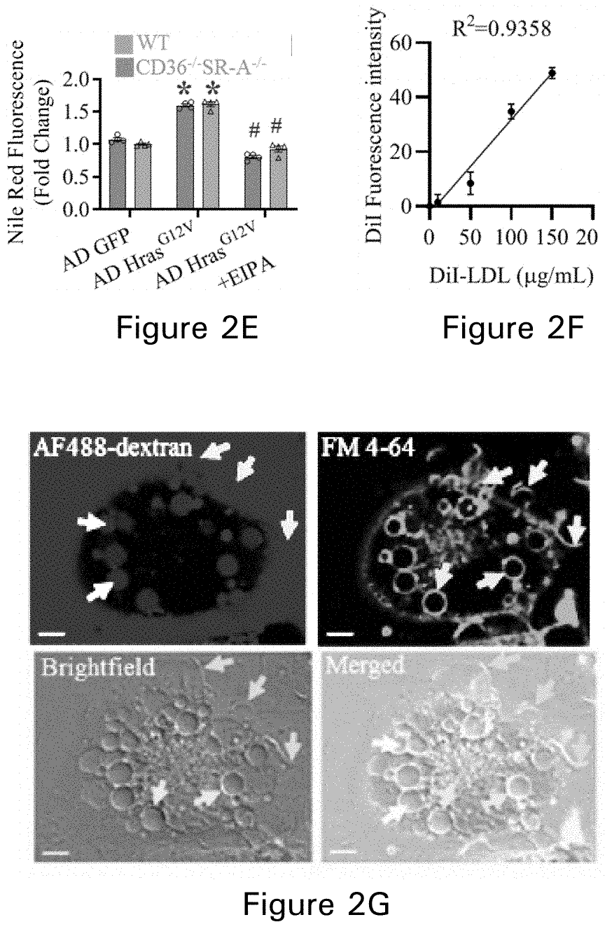

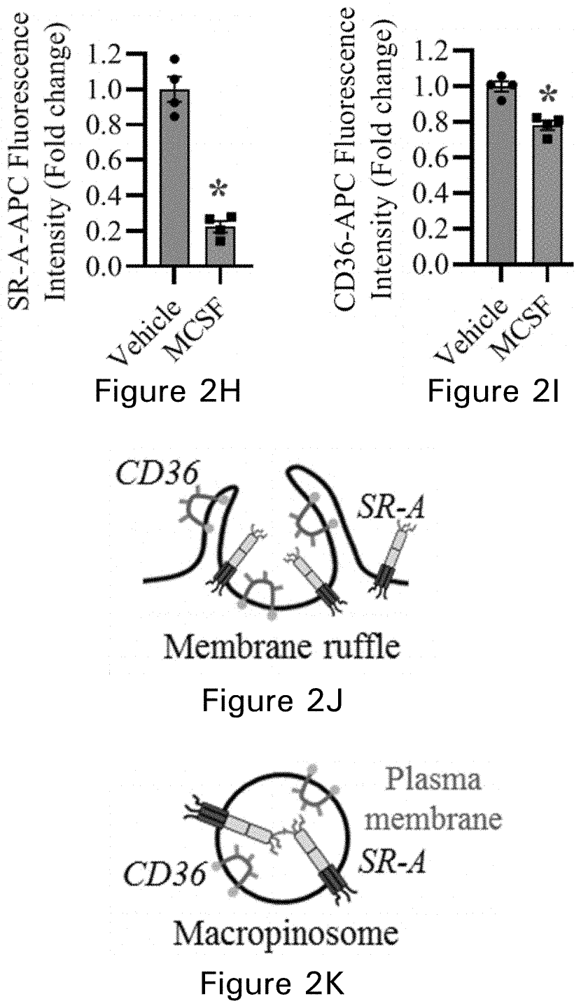

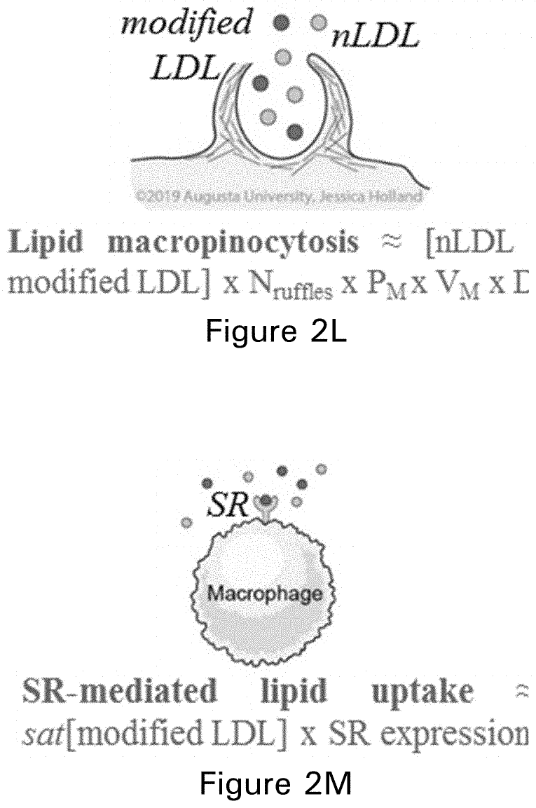

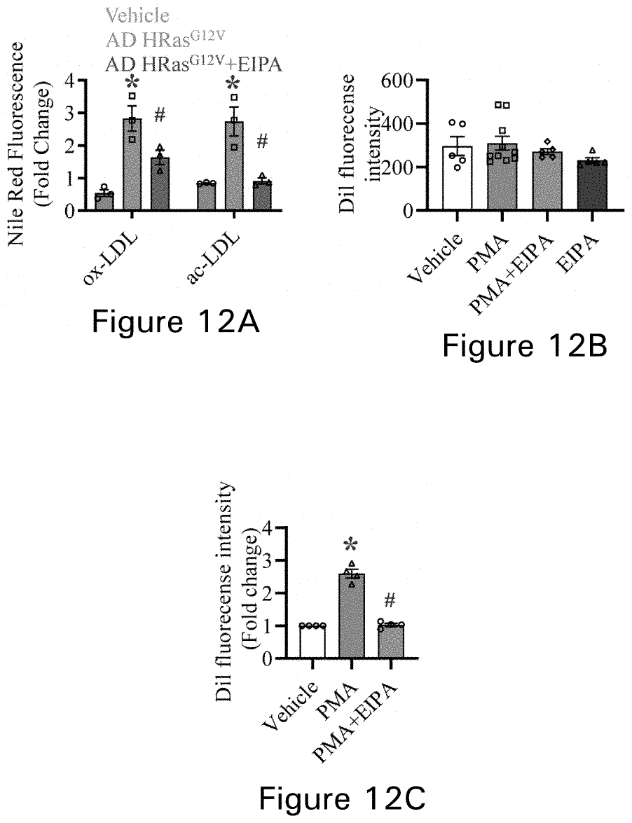

Growth factors stimulate macropinocytosis through activation of the small GTPase Ras, the production and turnover of phospholipids, and the tightly orchestrated rearrangements of the actin cytoskeleton in the submembranous layer of the cell. The highly dynamic reorganization of the actin cytoskeleton promotes formation of membrane ruffles in macrophages that may circularize and close, leading to cup formation and receptor-independent internalization of pericellular solutes via membrane-derived vesicles known as macropinosomes (FIG. 2A). Previous studies have compared uptake of modified vs. nLDL in macrophages under conditions that do not stimulate macropinocytosis (5). Accordingly, nLDL uptake was limited and the conclusion has been made that unmodified lipids are not atherogenic. To investigate the contribution of macropinocytosis-mediated vs. SR-dependent LDL uptake to foam cell formation, Ras (H-RAS^{G12V}) in WT and CD36^{-/-}/SR-A^{-/-} macrophages was overexpressed to be constitutively active to stimulate membrane ruffling (FIGS. 2B and C) and quantified internalization of nLDL, ox-LDL and ac-LDL using ORO staining and Nile Red fluorescence. As shown in FIGS. 2D&E, uptake of nLDL (50 μg/ml) by both WT and CD36^{-/-}/SR-A^{-/-} macrophages was significantly increased following stimulation of macropinocytosis. Importantly, overexpression of H-RAS^{G12V} in CD36^{-/-}/SR-A^{-/-} macrophages stimulated ox- and ac-LDL (50 μg/ml) internalization, leading to increased cholesterol accumulation (FIG. 12A). Preincubation of CD36^{-/-}/SR-A^{-/-} macrophages with EIPA inhibited Nile Red fluorescence suggesting that ox- and ac-LDL internalization is mediated by macropinocytosis (FIG. 12A). Control experiments demonstrate that EIPA does not promote cholesterol efflux in lipid-laden macrophages (FIG. 12B). These in vitro results may provide an explanation why atherosclerosis development is only incompletely inhibited in SR knockout mice.

### Example 3. Stimulation of Macropinocytosis Inhibits Plasma Membrane Expression of CD36 and SR-A

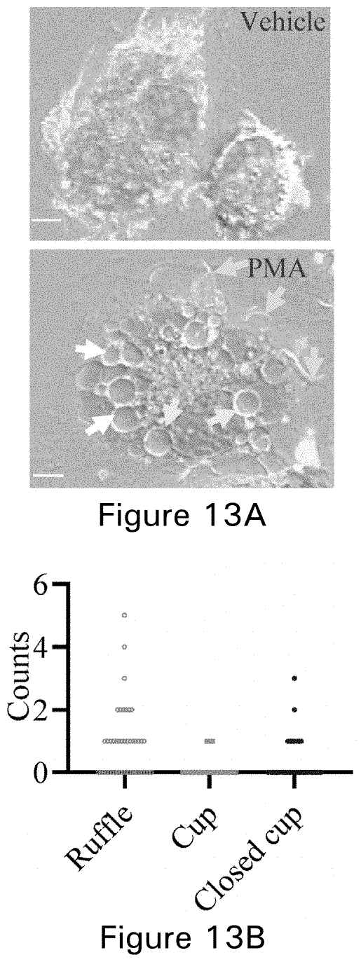

Macrophage internalization of fluorescently-labeled (DiI) nLDL by macropinocytosis is linearly related to extracellular lipid concentration (FIG. 2F and FIG. 12C). These results confirm that pericellular lipids do not require receptor binding in order to be internalized following stimulation of macropinocytosis. On the contrary, SR-mediated uptake of ox- and ac-LDL is saturated at concentrations of 25-50 μg/ml (34). Importantly, LDL concentration in human arteries typically exceeds 1,000-1,500 μg/ml suggesting that macropinocytosis could mediate substantially greater amount of macrophage cholesterol uptake compared with SR-mediated pathways of internalization. During macropinocytosis, macropinocytic cups close and pinch off creating macropinosomes that deliver not only extracellular lipids, but plasma membrane constituents into the cytosol (FM 4-64, arrows in FIG. 2G). Flow-cytometry analysis suggests that stimulation of macropinocytosis attenuates cell surface expression of CD36 and SR-A via macropinosome-mediated cytosolic internalization (FIGS. 2H-2K). Supporting these results, FIG. 2G, and FIGS. 13A and 13B, demonstrate internalization of fluorescently-labeled plasma membrane and encapsulation of extracellular solutes in macropinosomes (Alexa Fluor 488-dextran, white arrows) following macropinocytosis stimulation. Taken together, these results suggest that SR-mediated LDL uptake and lipid macropinocytosis are not mutually exclusive, they could operate independently or even regulate each other. The proposed equations describe and compare macropinocytosis- and SR-mediated lipid uptake in atherosclerotic arteries for the first time (FIGS. 2L and 2M):

Lipid macropinocytosis≈[nLDL+modified LDL]×N_{ruffles}×P_{M}×V_{M}×D_{M}

[nLDL+modified LDL]: native and modified LDL concentration in the artery

N_{ruffles}: Number of ruffles

P_{M}: Probability of macropinosome formation from existing ruffles

V_{M}: Volume of internalized macropinosomes

D_{M}: Duration of macropinocytosis

SR-mediated lipid uptake≈sat[modified LDL]×SR expression

d[modified LDL]: saturating concentration of modified LDL when reaches plateau

SR expression: SR expression in the plasma membrane

### Example 4. Physiological Stimulation of Macropinocytosis in Human and Murine Macrophages

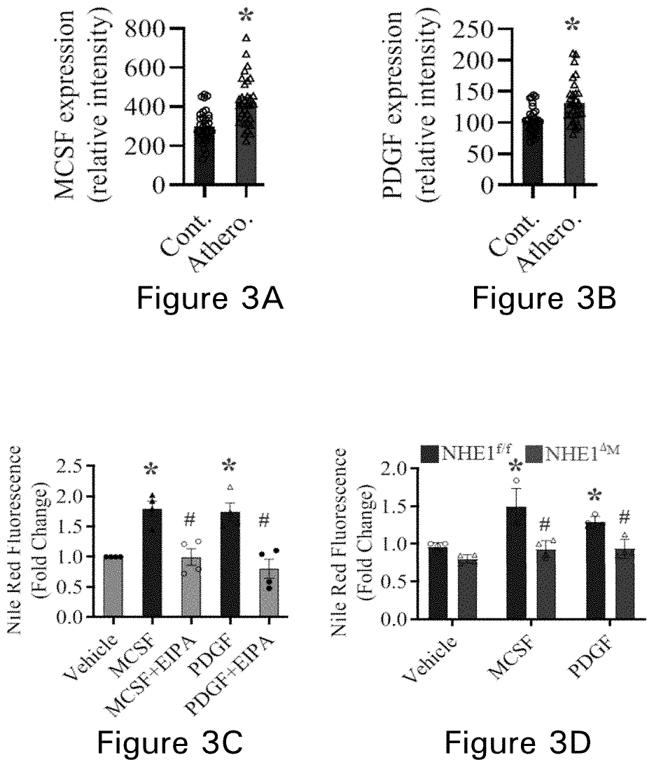

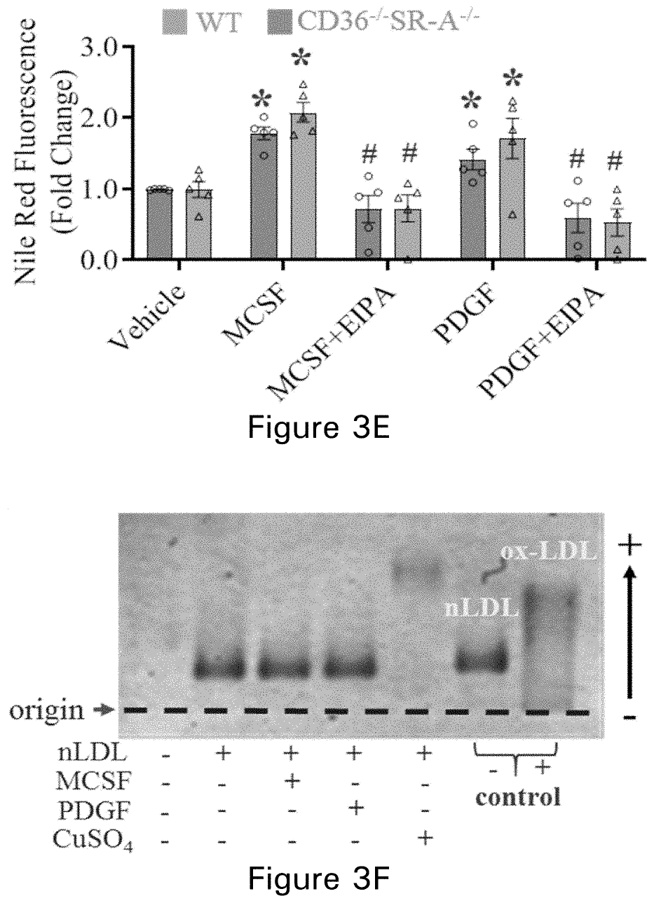

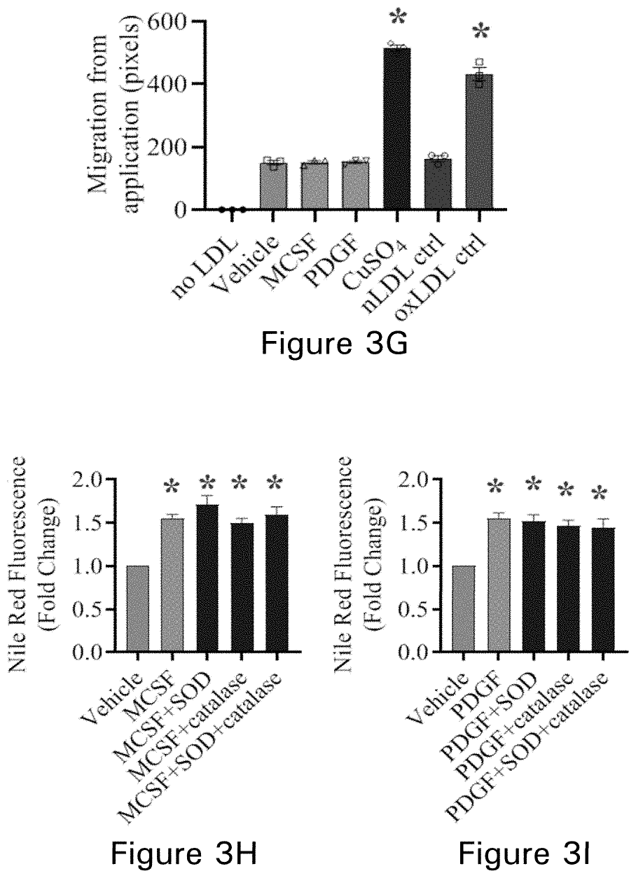

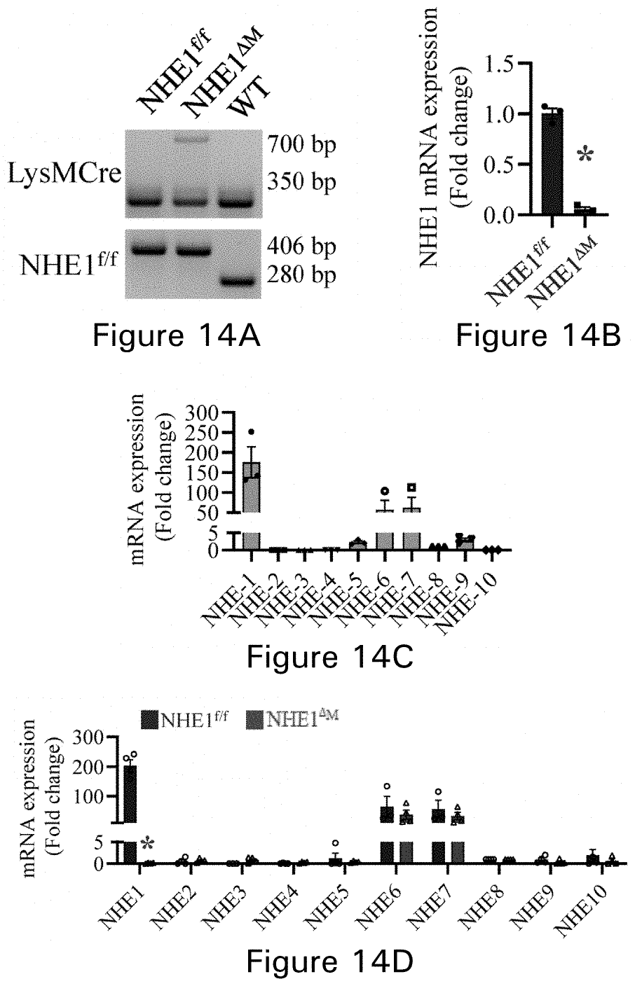

Lipid internalization by human macrophages in response to physiologically relevant stimulators of macropinocytosis that are upregulated in atherosclerotic arteries was investigated, including platelet-derived growth factor (PDGF) and macrophage colony-stimulating factor (M-CSF). Analysis of publicly available patient cohorts (Gene Expression Omnibus) confirmed increased expression of PDGF and M-CSF in human atherosclerotic vessels compared to non-atherosclerotic control tissue (n=32; FIGS. 3A and 3B). As shown in FIG. 3C, incubation of human THP-1 macrophages with PDGF and M-CSF stimulated macropinocytosis of nLDL and increased Nile Red fluorescence. Mice lacking NHE1 selectively in myeloid cells (LysMCre₊ NHE1^{fl/fl}, hereafter referred to as NHE1^{ΔM}) to inhibit macrophage macropinocytosis in vitro and in vivo (FIGS. 14A and 14B) were created. RT-PCR data demonstrate that NHE1 is the most highly expressed NHE isoform in macrophages and deletion of NHE1 in NHE1^{ΔM} mice did not induce compensatory changes in the expression of other NHE isoforms in macrophages (FIGS. 14C and 14D). As shown in FIG. 3D, PDGF- and M-CSF-induced nLDL internalization was inhibited in NHE1^{ΔM} bone marrow-derived macrophages (BMDM) compared with NHE1^{fl/fl} controls. Importantly, physiological stimulation of macropinocytosis resulted in increased uptake of nLDL by CD36^{-/-}/SR-A^{-/-} macrophages (FIG. 3E). Experiments were performed to examine whether M-CSF and PDGF oxidize nLDL in the media of cultured macrophages using agarose gel electrophoresis. The relative electrophoretic mobility (REM) of LDL from conditioned media of vehicle-, M-CSF- and PDGF-treated macrophages was not different from negative control nLDL, indicating no oxidative modification of lipids (i.e. negative charge) in response to M-CSF and PDGF treatments (FIGS. 3F and 3G). Consistent with these results, non-pegylated (e.g., cell-impermeant) superoxide dismutase (SOD) and catalase did not inhibit M-CSF- and PDGF-induced nLDL internalization (FIGS. 3H and 3I).

### Example 5. Visualization of Macrophage Macropinocytosis in Human and Murine Atherosclerotic Arteries

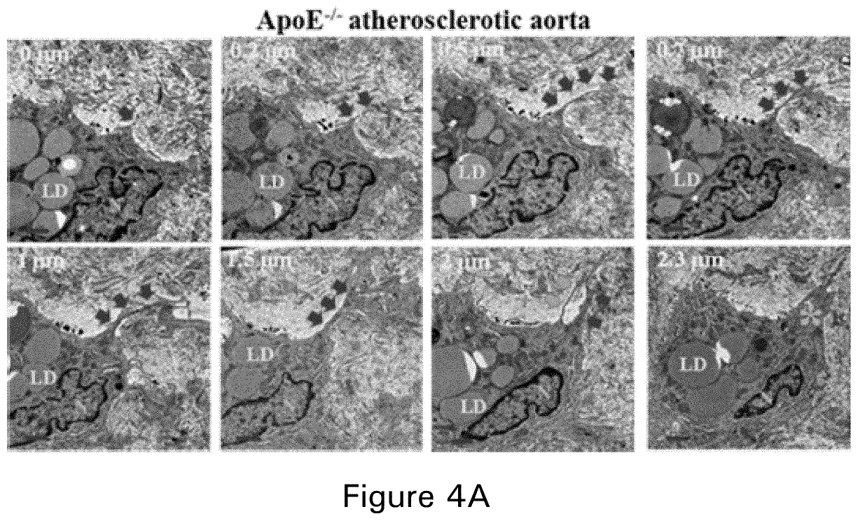

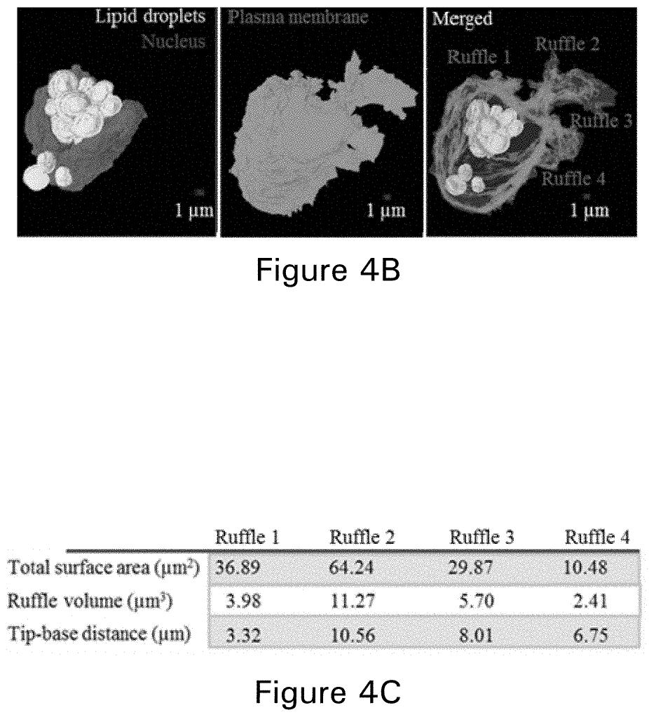

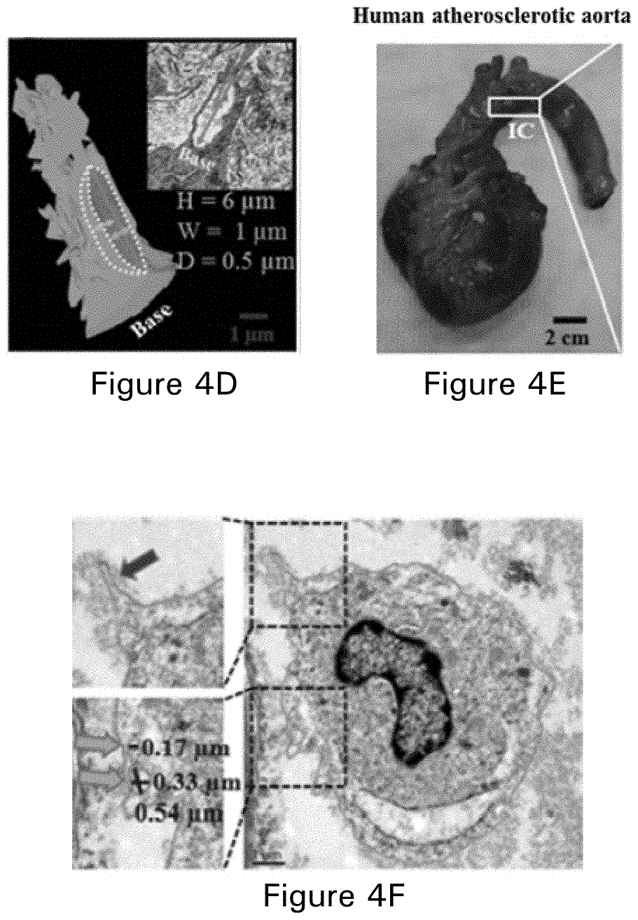

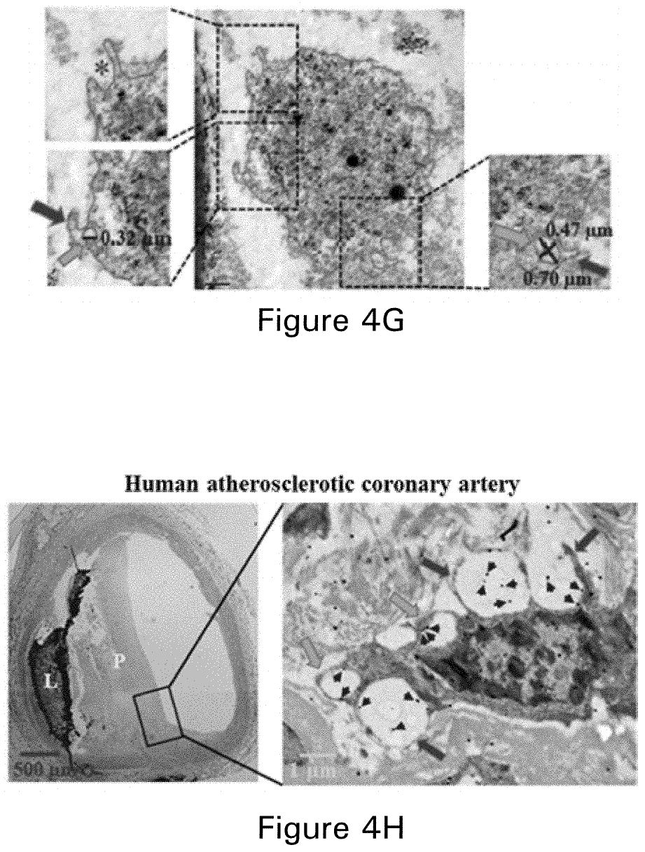

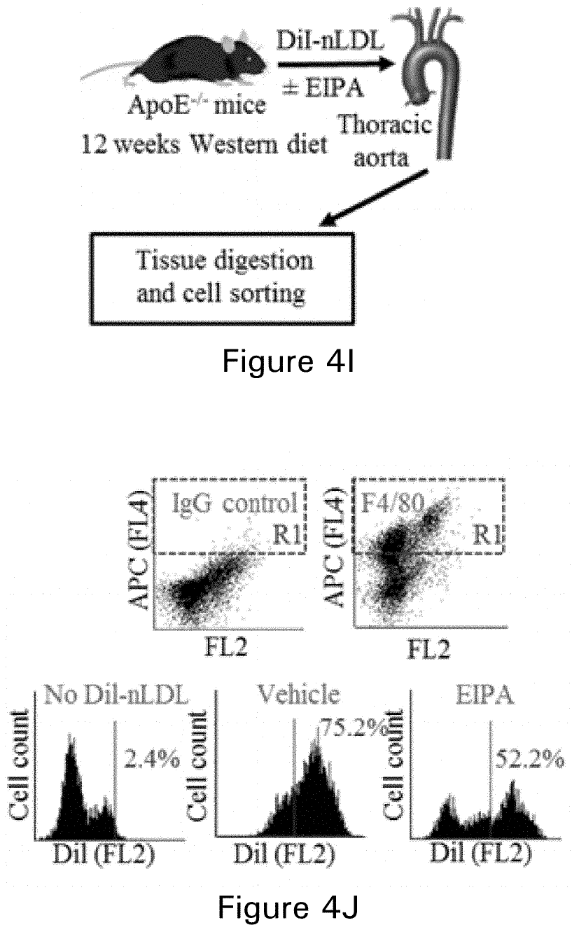

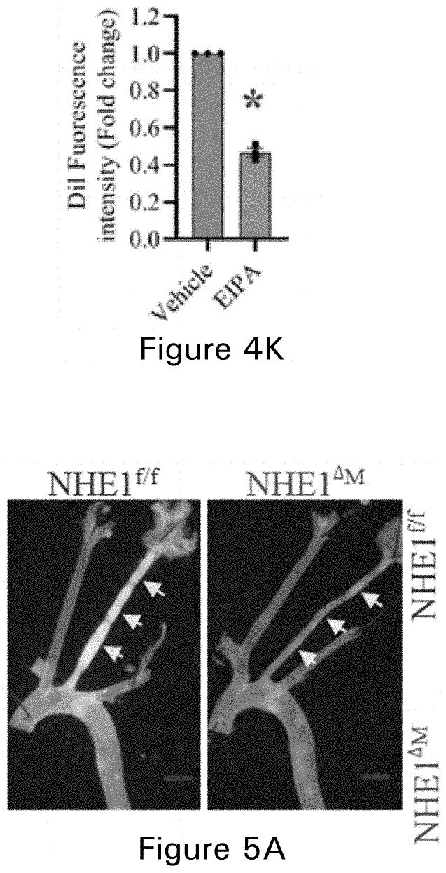

No previous studies have provided direct visual evidence of macrophage macropinocytosis in atherosclerotic vessels. Serial section Transmission Electron Microscopy (ssTEM) imaging was used to analyze the 3D ultrastructure of the subendothelial layer in atherosclerotic arteries. For these experiments, lipid-laden macrophages in the atherosclerotic aorta of ApoE^{-/-} mice (12 weeks Western diet) were followed through ˜200 ultra-thin (60 nm) serial sections. Analysis of subendothelial ultrastructure identified macrophage foam cells that develop large, sheet-like membrane protrusions (red arrows) that curve back (blue asterisk) to form parallel membrane protrusions (orange arrows) resembling circularized C-shaped ruffles (FIG. 4A). Three-dimensional reconstruction of ssTEM images provides additional details on the physical characteristic of membrane ruffles, including total surface area, ruffle volume, and tip-base distance (FIGS. 4B-4C). Reconstructed curved ruffles in 3D and corresponding 2D ssTEM images on the surface of macrophage foam cells are shown in FIG. 4D. Quantification of membrane ruffles, macropinocytic cups and cup closure in atherosclerotic arteries is shown in FIG. 13B. Next, human atherosclerotic arteries were used to provide translational relevance. Importantly, ssTEM images identified macrophages in the subendothelial layer of human atherosclerotic aorta that demonstrate the full cycle of macropinocytosis, including single membrane protrusions (red arrows), C-shaped ruffles or macropinocytotic cups (red asterisk), and formation of membrane-derived vesicles, ranging from 170 nm to 700 nm in diameter, located at the base of membrane protrusions in the cytosol (orange arrows) (FIGS. 4E-4G). Characterization of tissue donors, including age, sex, cardiovascular risk factors, medication, co-morbidities and cause of death is shown in Table 2. It is important to note that the size of clathrin- and caveolin-coated vesicles is more uniform and typically less than 100 nm in diameter, while macropinosomes are more heterogeneous in size and larger [150 nm to 5 μm in diameter].

TABLE 2
Medical information

| Patient # | Age | Sex | COD | Medical history | Medications | Risk Factors |
|---|---|---|---|---|---|---|
| 1 | 51 | F | Acute | Type 2 diabetes | Albuterol, Aspirin, | Hypertension, |
| | | | respiratory | mellitus, | Glipizide, | dyslipidemia |
| | | | failure | Hypertension | Hydrochlorothiazide, | and |
| | | | | Atherosclerosis | Levemir, Lisinopril, | hyperglycemia |
| | | | | Chronic renal failure | Metformin, Naproxen | |
| | | | | Obesity | | |
| 2 | 57 | F | Hypertension | End-stage renal | Aspirin, Atorvastatin, | Hypertension, |
| | | | and | disease, Chronic | Calcitriol, Carvedilol, | dyslipidemia, |
| | | | atherosclerosis | obstructive | Ferrous sulfate, Folic | hyperglycemia, |
| | | | | pulmonary disease, | acid, Haloperidol, | smoking, |
| | | | | cardiac arrhythmia, | Hydrazine, | alcoholism |
| | | | | Coronary Artery | Metoprolol, | |
| | | | | Bypass Grafting, | Mirtazapine, | |
| | | | | Alcohol, tobacco and | Nitroglycerin, | |
| | | | | drug abuse | Phoslyra, Protonix, | |
| | | | | | Sevelamer, Tramadol, | |
| | | | | | Vitamin B1, | |
| | | | | | Wellbutrin, Zoloft | |

COD—cause of death

Impaired efferocytosis of apoptotic cells has been demonstrated to contribute to the pathogenesis of atherosclerosis. Solid particles, microbes or fragments of apoptotic cells were not visualized near the plasma membrane of macrophages, indicating efferocytosis-independent plasma membrane activities (FIGS. 4F and 4G). As coronary artery atherosclerosis is the leading cause of death in both men and women in developed countries, human atherosclerotic left anterior descending (LAD) coronary arteries [type Vb lesion] from cadaveric donors (FIG. 4H) were sectioned and Immunoelectron Microscopy (IEM) imaging was performed to detect LDL in macrophage-rich areas. Immunolabeling localized LDL in the cytosol of subendothelial macrophages and near the plasma membrane, within open macropinocytic cups (red arrows) and closed macropinosomes (orange arrows) in human atherosclerotic LAD (FIG. 4H). Although aggregated LDL (agg-LDL) has been shown to induce phagocytosis in macrophages, diameter of phagocytic cups surrounding agg-LDL is expected to be in the lower nm range [50-300 nm] and significantly smaller than the observed cups that are 1 μm in diameter or larger.

No previous studies have investigated macrophage macropinocytosis of LDL in atherosclerotic arteries. Next, atherosclerotic ApoE^{-/-} mice (12 weeks Western diet) were treated with vehicle or EIPA (25 mg/kg/day, i.p. for 3 days prior to sacrifice) and retro-orbitally injected with DiI-nLDL (100 μg) to quantify lipoprotein macropinocytosis by macrophages in atherosclerotic lesions (FIG. 4I). Flow cytometry analysis demonstrated that isolated F4/80⁺ macrophages internalized high amounts of DiI-LDL in atherosclerotic arteries and the uptake was substantially inhibited by pharmacological inhibition of macropinocytosis (FIGS. 4J and 4K).

### Example 5. Genetic Inhibition of Macropinocytosis Selectively in Myeloid Cells Inhibits Atherosclerosis

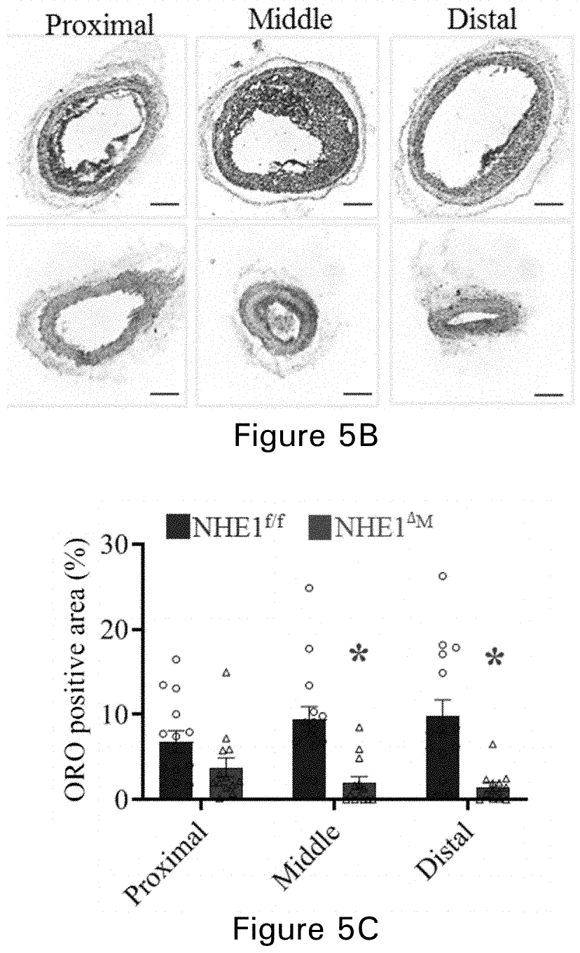

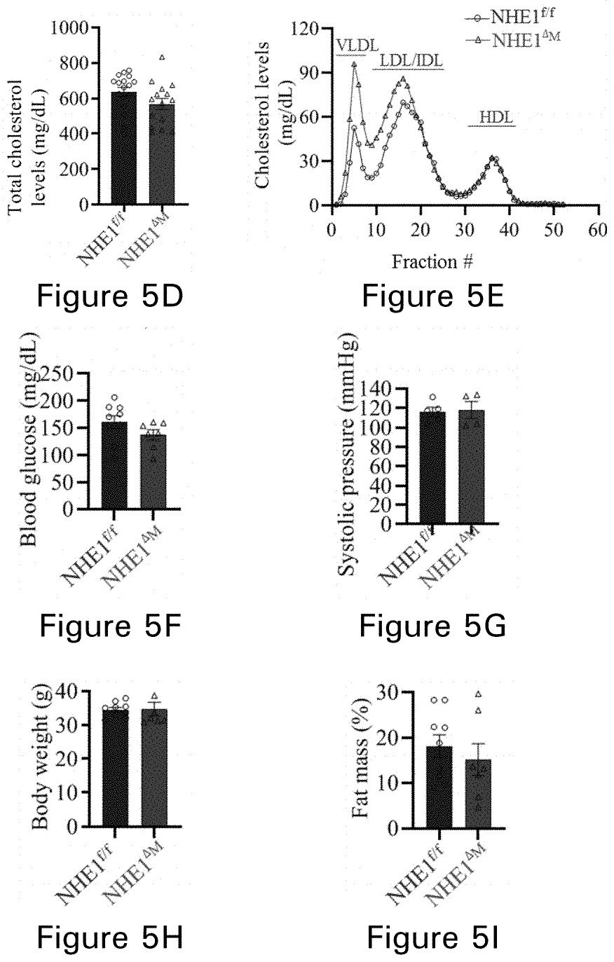

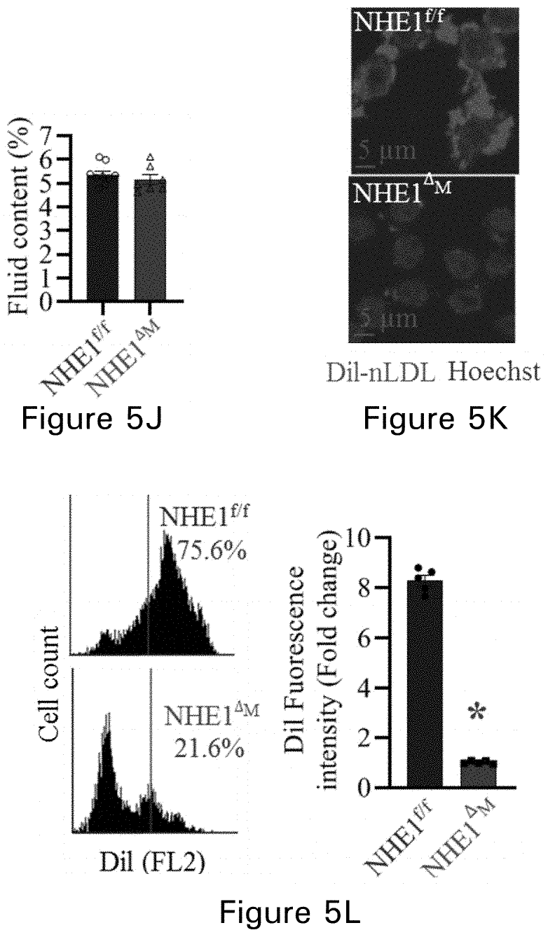

Mice with myeloid-specific deletion of NHE1 (NHE1^{ΔM}) were generated to inhibit macrophage macropinocytosis in vivo (FIG. 14A). Quantitative RT-PCR analysis of primary BMDM isolated from NHE1^{ΔM} mice showed 95% lower mRNA levels of NHE1 compared with NHE1^{f/f} macrophages (FIG. 14B). Flow cytometry analysis demonstrated that peritoneal NHE1-deficient macrophages internalized significantly lower amounts of exogenously administered DiI-nLDL compared to NHE1^{f/f} controls, confirming inhibition of macrophage macropinocytosis in NHE1^{ΔM} mice in vivo (FIGS. 5K and 5L). First, atherosclerosis was induced by AAV-mediated overexpression of PCSK9, partial LCA ligation and 4 weeks Western diet. No changes in body weight, fat mass, plasma cholesterol, glucose levels and blood pressure were observed (FIGS. 5D to 5J), but the NHE1^{ΔM} mice developed significantly smaller atherosclerotic lesions compared to NHE1^{f/f} controls (FIGS. 5A to 5C).

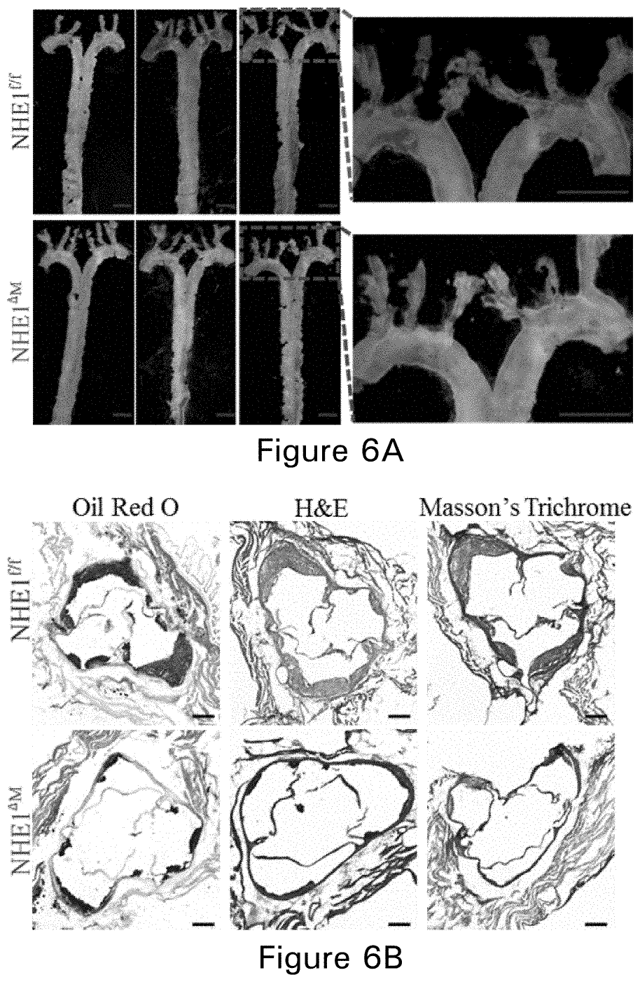

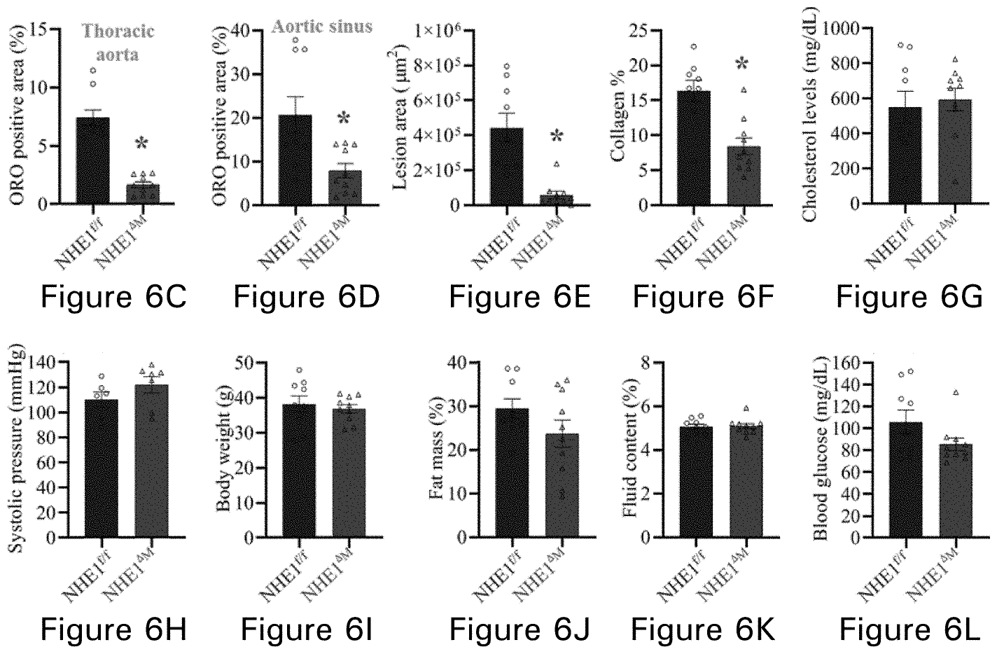

The LCA model represents a combination of vascular injury, disturbed flow and a relatively short-term hypercholesterolemia. To address this limitation and confirm results, a traditional atherosclerosis model of chronic hypercholesterolemia was used. The results demonstrated that atherosclerosis development is significantly decreased in the aortic sinus and entire thoracic aorta of NHE1^{ΔM} mice following 16 weeks of Western diet compared to control animals (FIGS. 6A to 6D). Collagen content, atherosclerotic lesion area and CD68⁺ macrophage staining were significantly decreased in the aorta of NHE1^{ΔM} mice compared with NHE1^{f/f} controls (FIGS. 6B, 6E, 6F and 6G). Plasma cholesterol levels, systolic blood pressure, body weight, fat mass, fluid content and fasting glucose levels in NHE1^{ΔM} mice were not different from control animals (FIGS. 6H to 6M). These results suggest that inhibition of macropinocytosis selectively in myeloid cells attenuates development of atherosclerosis in hypercholesterolemic mice

### Example 5. A “Repurposed” FDA-Approved Drug that Inhibits Macrophage Macropinocytosis Attenuates Atherosclerosis Development in Hypercholesterolemic Mice

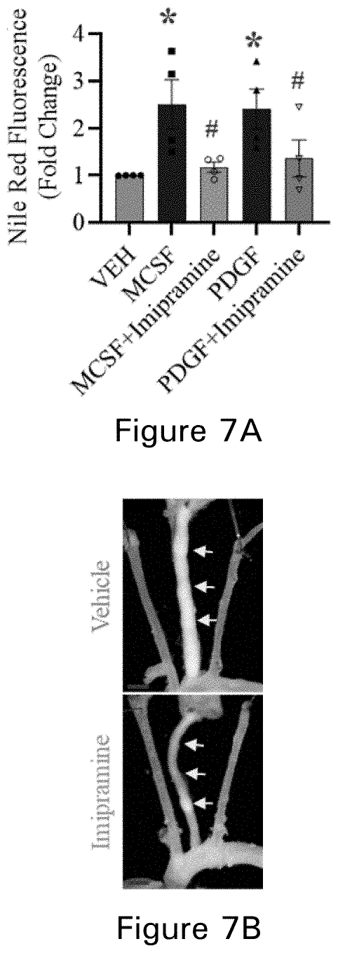

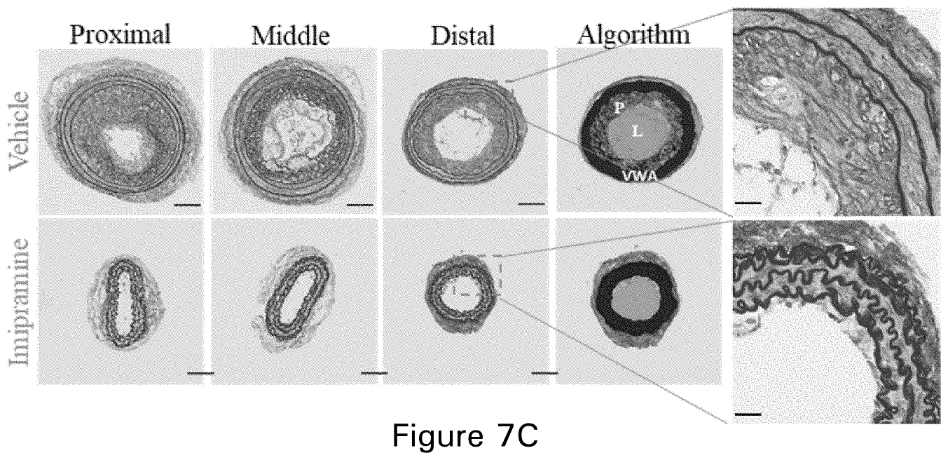

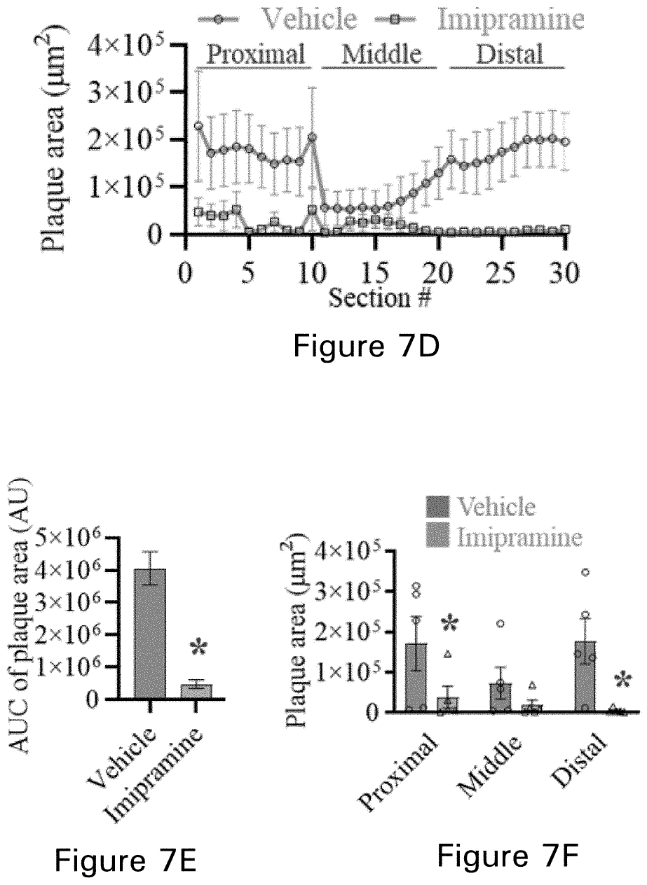

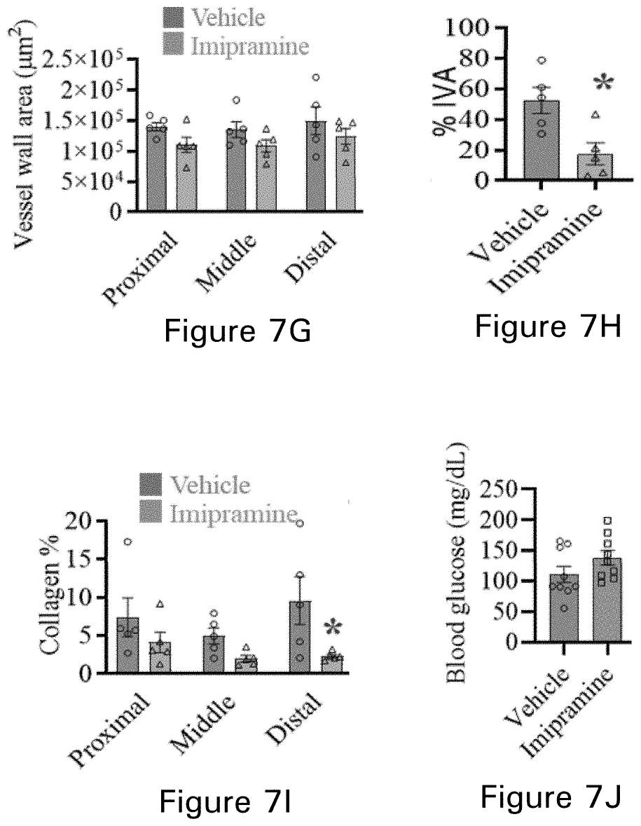

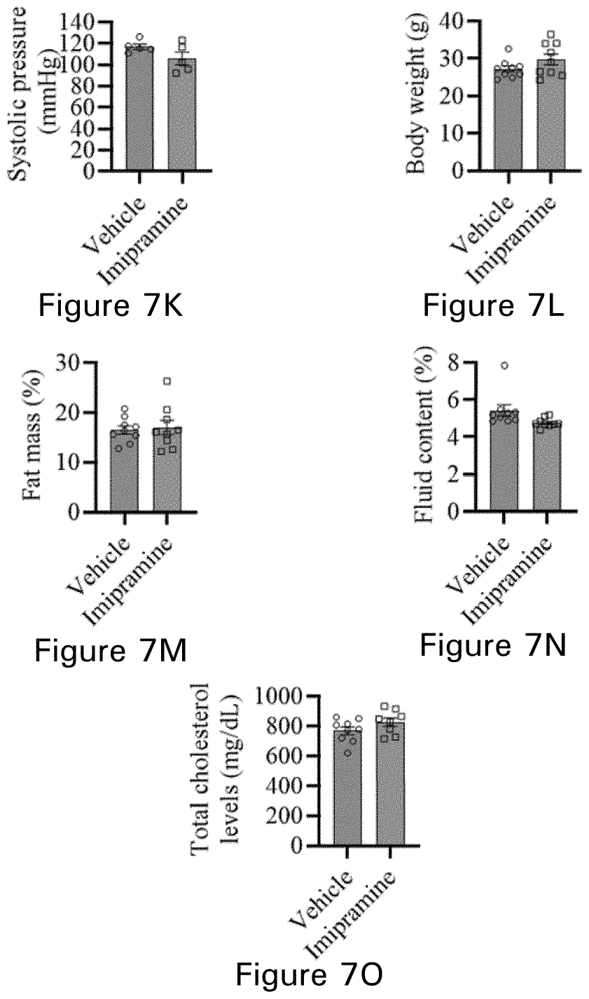

A previous study demonstrated that amiloride monotherapy improves pulse wave velocity (PWV), a surrogate marker for arterial stiffness and atherosclerosis, in pre-hypertensive patients independent of its blood pressure lowering effect. Despite this information, no prior studies have investigated whether pharmacological inhibition of macropinocytosis would be a viable therapeutic strategy for patients with atherosclerotic vascular disease. Although amiloride and its analogues that selectively block NHE1 are considered to be the best choices for pharmacological inhibition of macropinocytosis in animal models, possible effects of these drugs on transmembrane Na⁺ transport, pH regulation and water homeostasis, in addition to their capacity to block macropinocytosis, limit their use as therapeutic agents in cardiovascular medicine. The next goal, therefore, was to utilize a clinically relevant inhibitor of macrophage macropinocytosis that lacks regulatory effects on NHE1 and test its ability to inhibit atherosclerosis development. A large unbiased-screen of an FDA-approved drug library was recently performed and identified a potent (IC₅₀=131 nM), non-toxic [selectivity index (CC₅₀/IC₅₀)>300] low MW compound (imipramine) that selectively inhibits macropinocytosis in macrophages, independent of NHE1 regulation. Imipramine is a tricyclic antidepressant (TCA) with high oral bio-availability (95%) and a half-life of nearly 20 hours that is clinically used in children to treat enuresis and adults with depression. As shown in FIG. 7A, preincubation of macrophages with imipramine inhibited M-CSF- and PDGF-induced intracellular lipid accumulation in macrophages following nLDL treatment. The efficacy of imipramine (9 mg/mL, pump rate: 0.11 μl/hour, sc) to attenuate atherosclerosis development in hypercholesterolemic mice was investigated. Combined Orcein and Martius Scarlet Blue (OMSB) staining followed by automatic segmentation and pseudocoloring (FIG. 7C; Algorithm) was used for qualitative and quantitative analyses of atherosclerotic lesions as described previously. Results from OMSB staining demonstrated that imipramine significantly attenuates atherosclerotic lesion formation compared with vehicle treatment in LDLR-knockdown mice (FIGS. 7B to 7G). Imipramine inhibited relative internal vessel area (IVA %) and collagen area (collagen %) compared with vehicle treatment (FIGS. 7H and 7I). Furthermore, no significant differences were observed in total cholesterol levels, blood pressure, body weight, fat mass and whole body fluid content between imipramine-treated and control mice (FIGS. 7J to 7O). Taken together, the results of these experiments support the use of repurposed macropinocytosis inhibitors for pharmacological treatment of atherosclerosis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for treating atherosclerotic vascular disease in a subject in need thereof comprising:
administering to the subject an effective amount of a pharmaceutical composition comprising one or more inhibitors of macropinocytosis to inhibit or reduce receptor-independent LDL macropinocytosis in the subject.

2. The method of claim 1, wherein the one or more inhibitors of macrophage macropinocytosis comprises imipramine or a derivative thereof or any additional macropinocytosis inhibitors independent of their effects on NHE1 activity.

3. The method of claim 2, wherein the macropinocytosis is macrophage macropinocytosis.

4. The method of claim 1, wherein the atherosclerotic vascular disease is atherosclerosis.

5. A method for reducing plaque formation in a subject's circulatory system in need thereof comprising:
administering to the subject an effective amount of a pharmaceutical composition comprising one or more inhibitors of macropinocytosis to inhibit or reduce receptor-independent LDL macropinocytosis, reduce plaque formation or induce atherosclerosis regression in the subject's circulatory system.

6. The method of claim 5, wherein the one or more inhibitors of macrophage macropinocytosis comprises imipramine or a derivative thereof or any additional macropinocytosis inhibitors independent of their effects on NHE1 activity.

7. The method of claim 6, wherein the macropinocytosis is macrophage macropinocytosis.

8. The method of claim 1, wherein the subject has atherosclerosis.

9. A method for reducing or inhibiting foam cell formation in a subject in need thereof comprising:
administering to the subject an effective amount of a pharmaceutical composition comprising one or more inhibitors of macropinocytosis to inhibit or reduce receptor-independent LDL macropinocytosis to inhibit or reduce foam cell formation in the subject.

10. The method of claim 10, wherein the one or more inhibitors of macrophage macropinocytosis comprises imipramine or a derivative thereof or any additional macropinocytosis inhibitors independent of their effects on NHE1 activity.

11. The method of claim 10, wherein the macropinocytosis is macrophage macropinocytosis.

12. The method of claim 10, wherein the subject has atherosclerosis.

13. A method for treating atherosclerotic vascular disease in a subject in need thereof comprising:
administering to the subject an effective amount of a pharmaceutical composition comprising one or more inhibitors of macropinocytosis to inhibit or reduce receptor-independent LDL macropinocytosis in the subject in combination or alternation with one or more statins, other lipid lowering agents or cardiovascular therapeutics.

14. The method of claim 13, wherein the one or more inhibitors of macrophage macropinocytosis comprises imipramine or a derivative thereof or any additional macropinocytosis inhibitors independent of their effects on NHE1 activity and the one or more statins are selected from the group consisting of atorvastatin, fluvastatin, lovastatin, pravastatin, rosuvastatin, simvastatin, pitavastatin, and combinations thereof.

15. A pharmaceutical composition comprising:
an effective amount of one or more inhibitors of macropinocytosis; and
an effective amount of one or more statins, other lipid lowering agents or cardiovascular therapeutics.

16. The pharmaceutical composition of claim 15, wherein the one or more inhibitors of macrophage macropinocytosis comprises imipramine or a derivative thereof or any additional macropinocytosis inhibitors independent of their effects on NHE1 activity and the one or more statins are selected from the group consisting of atorvastatin, fluvastatin, lovastatin, pravastatin, rosuvastatin, simvastatin, pitavastatin, and combinations thereof.

17. The pharmaceutical composition of claim 16, wherein the pharmaceutical composition is formulated for oral administration.
